(19) United States
(12) Patent Application Publication
(10) Pub. No.: US 2026/0256720 A1
(43) Pub. Date: Sep. 3, 2026

(54) ACETAMINOPHEN COMPOSITIONS AND METHODS OF TREATING CANCER

(71) Applicants: The United States Government as represented by the Department of Veterans Affairs, Washington, DC (US); Virginia Commonwealth University, Richmond, VA (US)

(72) Inventors: Alexander Neuwelt, Richmond, VA (US); Allyn Bryan, Richmond, VA (US)

(21) Appl. No.: 19/163,963

(22) PCT Filed: Mar. 14, 2024
(86) PCT No.: PCT/US2024/019873

§ 371 Date: Sep. 10, 2025

Related U.S. Application Data
(60) Provisional application No. 63/490,230, filed on Mar. 14, 2023.

(51) Int. Cl. A61K 31/167; A61K 9/08; A61K 31/047; A61K 31/10; A61K 31/136; A61K 31/145; A61K 31/18; A61K 31/415; A61K 31/416; A61K 31/4174; A61K 31/4409; A61K 47/02; A61K 47/18; A61K 47/24; A61P 35/00

(52) U.S. Cl. CPC A61K 31/167; A61K 9/08; A61K 31/047; A61K 31/10; A61K 31/136; A61K 31/145; A61K 31/18; A61K 31/415; A61K 31/416; A61K 31/4174; A61K 31/4409; A61K 47/02; A61K 47/183; A61K 47/24; A61P 35/00

(57) ABSTRACT

The disclosure relates to compositions including acetaminophen or analogs thereof and CYP2E1-inhibitors suitable for parenteral or oral administration. Methods of treating cancer in subjects by administering acetaminophen or analogs thereof and CYP2E1-inhibitors are also included.

### CROSS REFERENCE TO RELATED APPLICATIONS

This application claims the benefit of U.S. Provisional Application No. 63/490,230, filed Mar. 14, 2023. The content of this earlier filed application is hereby incorporated by reference herein in its entirety.

### GOVERNMENT SUPPORT

This invention was made with government support under grant number 5IK2BX004914-02 awarded by the Department of Veterans Affairs. The government has certain rights in this invention.

### BACKGROUND

In the United States, lung cancer has an incidence of 225,000 patients every year leading to approximately 160,000 deaths (Siegel R L, et al. C A Cancer J Clin. 2017; 67 (1): 7-30). Over the last several years, the development of new therapies, including targeted therapies for patients with driver mutations in genes such as EGFR and ALK, have revolutionized the care of patients with lung cancer. Nevertheless, the vast majority of non-small cell lung cancer (NSCLC) patients either don't respond (due to lack of a targetable driver mutation) or develop resistance to these therapeutic options. As a result, chemotherapy continues to be a mainstay in the management of patients with NSCLC. While many patients with metastatic NSCLC initially respond to chemotherapy, the remission tends to be short-lived, and tumor regrowth is almost inevitable. This re-growth is widely felt to be secondary to a sub-population of tumor cells, termed CSCs, that are capable of self-renewal, tumor initiation, and de-differentiation (Visvader J E and Lindeman G J. Nat Rev Cancer. 2008; 8 (10): 755-68). CSCs are resistant to chemotherapy and give rise to difficult to treat recurrent cancer (Visvader J E and Lindeman G J. Nat Rev Cancer. 2008; 8 (10): 755-68). Data is similarly emerging that driver mutation-positive NSCLC tumor cells that develop resistance to tyrosine kinase inhibitor (TKI) therapy similarly exhibit a CSC phenotype (Shao Y, et al. Oncol Lett. 2018; 15 (5): 6655-65; and Honkanen T, et al. Int J Oncol. 2017; 51 (2): 599-606).

Extensive research suggests that, in addition to regulating cell growth and proliferation, angiogenesis, and malignant transformation (Yu H, et al. Nat Rev Cancer. 2014; 14 (11): 736-46), signaling pathways mediated by the STAT3 transcription factor are activated preferentially in CSCs. STAT3 signaling is important for the growth, proliferation and maintenance of CSCs (Wei W, et al. Stem Cells. 2014; 32 (10): 2571-82; and Marotta L L, et al. J Clin Invest. 2011; 121 (7): 2723-35), and phosphorylated STAT3 expression is associated with poor prognosis in lung cancer (Sun Z G, et al. Oncol Lett. 2018; 15 (4): 4278-88). Indeed, STAT3 is important for the maintenance of aldehyde dehydrogenase positive CSCs in non-small cell lung cancer (NSCLC) (Shao C, et al. Clin Cancer Res. 2014; 20 (15): 4154-66).

Phosphorylation of STAT3 at Y705 leads to SH2 domain mediated dimerization and translocation into the nucleus. Within the nucleus, STAT3 binds to promoter sequences thereby modulating transcriptional expression of genes that regulate cellular proliferation (cyclin D1), angiogenesis (VEGF), apoptosis (Bcl-XL), and CSC regulators (e.g., CD133 (Ghoshal S, et al. Hepatobiliary Surg Nutr. 2016; 5 (3): 201-3)) among others (Yu H, et al. Nat Rev Cancer. 2014; 14 (11): 736-46). While the phosphorylation event at Y705 plays an important role in modulating STAT3 transcriptional activity, STAT3 is a pleiotropic protein with multiple intracellular functions (Yu H, et al. Nat Rev Cancer. 2014; 14 (11): 736-46) including the regulation of mitochondrial oxidative metabolism (Wegrzyn J, et al. Science. 2009; 323 (5915): 793-7).

Due to the known tumorigenic role of the STAT3 transcription factor, extensive efforts have been undertaken to develop selective inhibitors of STAT3 phosphorylation. Unfortunately, there currently are no STAT3 inhibitors FDA approved for the management of cancer patients. One limitation faced by STAT3 inhibitors in clinical development is a relative lack of specificity for STAT3 over STAT1 (Jung K H, et al. Clin Cancer Res. 2017; 23 (18): 5537-46)). Specificity for STAT3 over STAT1 is desirable because STAT3 appears to be pro-tumorigenic and STAT1 plays an important role in the anti-tumor immune response (Avalle L, et al. JAKSTAT. 2012; 1 (2): 65-72)). Achieving specificity of STAT3 over STAT1 is challenging due to the high degree of homology between the proteins (53% identical and 72% similar) (Trilling M, et al. J Immunol. 2014; 192 (1): 447-58).

Other STAT3 inhibitors in clinical development are rationally designed to eliminate STAT3 protein expression—as opposed to modulating STAT3 function via inhibition of STAT3 phosphorylation. Examples of such approaches include the use of anti-sense oligonucleotides (Reilley M J, et al. J Immunother Cancer. 2018; 6 (1): 119) and small molecules that selectively target STAT3 protein for ubiquitination (Bai L, et al. Cancer Cell. 2019; 36 (5): 498-511 e17). However, eliminating STAT3 expression may not be suited for the management of lung cancer patients. Total STAT3, unlike pSTAT3, is not associated with poor prognosis in patients with lung cancer (Sun Z G, et al. Oncol Lett. 2018; 15 (4): 4278-88), and lung adenocarcinoma patients with low STAT3 mRNA expression have improved prognosis (Galoczova M, et al. Cell Mol Biol Lett. 2018; 23:12). Further, complete ablation of STAT3 may have substantial toxicities; STAT3 plays critical physiological roles in many organ systems including the nervous system, skin, and immune system (Levy D E and Lee C K. J Clin Invest. 2002; 109 (9): 1143-8).

Lung cancer is the second most common cancer diagnosis of patients within the Veterans Affairs system, accounting for 19% of diagnosed malignancies (Zullig L L, et al. Mil Med. 2012; 177 (6): 693-701). Even though cancers tend to be identified at earlier stages within the VA system relative to the US population as a whole, about 23% of lung cancers diagnosed at VA hospitals are localized at diagnosis (Zullig L L, et al. Mil Med. 2012; 177 (6): 693-701). The 5-year survival rate among patients with metastatic lung cancer is about 6%. As a result lung cancer is the leading cause of cancer-related mortality in the United States (Goldstraw P, et al. J Thorac Oncol. 2016; 11 (1): 39-51) leading to 150,000 deaths in 2018 alone (Siegel R L, et al. C A Cancer J Clin. 2018; 68 (1): 7-30). Among veterans, the incidence of lung cancer is higher, and the survival rates are lower relative to the civilian population (Campling B G, et al. Cancer. 2005; 104 (4): 833-40). The use of high dose acetaminophen (AAP) as an anti-cancer agent has been studied both pre-clinically and clinically. In a phase I clinical trial involving a variety of cancer histologies, single agent high dose AAP demonstrated preliminary evidence of anti-cancer activity. AAP was followed by delayed rescue with the anti-oxidant N-acetylcysteine (NAC), the established antidote for AAP overdose-induced liver toxicity, and no dose limiting toxicity was observed despite treating patients with up to 20 g/m² AAP. The most profound responses were observed in patients with aero-digestive malignances (Kobrinsky N L, et al. Cancer Invest. 1996; 14 (3): 202-10). Subsequently, a 3-year old patient with hepatoblastoma that was cisplatin-refractory and had failed two prior therapies was treated with AAP 30 g/m² plus cisplatin with delayed NAC rescue. The patient had a durable remission and was disease free seven years later at the time his case was published (Kobrinsky N L, et al. Pediatr Blood Cancer. 2005; 45 (2): 222-5).

### SUMMARY

Disclosed herein are compositions and methods of administering compositions that permit the administration of acetaminophen in subjects where acetaminophen would not otherwise be effective (e.g., at normal or maximal safe dosages) and prevent the need to administer to the subject other pain medicines that can be highly addictive or are associated with an increase risk of an overdose or death such as an opioid (e.g., hydrocodone, oxycodone, oxymorphone, morphine, codeine, and fentanyl).

Also disclosed herein are compositions and methods of administering compositions that permit the administration of acetaminophen in subjects at doses of acetaminophen that are higher than standard doses of acetaminophen for treating cancer with an improved toxicity profile.

Disclosed herein are compositions comprising: acetaminophen or an analog thereof, and CYP2E1 inhibitor.

Disclosed herein are pharmaceutical compositions comprising: acetaminophen or an analog thereof; and a CYP2E1 inhibitor.

Disclosed herein are methods reducing or inhibiting growth of a tumor in a subject, the method comprising administering to the subject a therapeutically effective amount of the composition comprising: acetaminophen or an analog thereof; and CYP2E1 inhibitor or the pharmaceutical composition comprising: acetaminophen or an analog thereof; and CYP2E1 inhibitor.

Disclosed herein are methods of reducing or inhibiting growth of a tumor in a subject, the methods comprising administering to the subject a therapeutically effective amount of acetaminophen or an analog thereof and a therapeutically effective amount of a CYP2E1 inhibitor.

Disclosed herein are methods of inducing apoptosis of a cancer cell, the methods comprising administering to a subject a therapeutically effective amount of the composition comprising: acetaminophen or an analog thereof; and CYP2E1 inhibitor or the pharmaceutical composition comprising: acetaminophen or an analog thereof; and CYP2E1 inhibitor.

Disclosed herein are methods of inducing apoptosis of a cancer cell, the methods comprising administering to the subject a therapeutically effective amount of acetaminophen or an analog thereof and a therapeutically effective amount of a CYP2E1 inhibitor.

Disclosed herein are methods of treating cancer in a subject, the methods comprising administering to the subject a therapeutically effective amount of the composition comprising: acetaminophen or an analog thereof; and CYP2E1 inhibitor or the pharmaceutical composition comprising: acetaminophen or an analog thereof; and CYP2E1 inhibitor.

Disclosed herein are methods of treating cancer in a subject, the methods comprising administering to the subject a therapeutically effective amount of acetaminophen or an analog thereof and a therapeutically effective amount of a CYP2E1 inhibitor.

Disclosed herein are methods of reducing, preventing or treating pain in a subject, the methods comprising administering to the subject a therapeutically effective amount of the composition comprising: acetaminophen or an analog thereof; and CYP2E1 inhibitor or the pharmaceutical composition comprising: acetaminophen or an analog thereof; and CYP2E1 inhibitor.

Disclosed herein are methods of reducing, preventing or treating fever in a subject, the methods comprising administering to the subject a therapeutically effective amount of the composition comprising: acetaminophen or an analog thereof; and CYP2E1 inhibitor or the pharmaceutical composition comprising: acetaminophen or an analog thereof; and CYP2E1 inhibitor.

### BRIEF DESCRIPTION OF THE DRAWINGS

FIGS. 1A-C show N-acetylcysteine (NAC) co-administered with fomepizole prevents acetaminophen (AAP) toxicity. Mice were untreated or treated with AAP at indicated dose (mg/kg), NAC (100 mg/kg), sodium thiosulfate (STS, 3 g/kg), fomepizole (fom, 50 mg/kg) and sacrificed the following day. The serum was analyzed for ALT (FIG. 1A) and BUN (FIG. 1B), and the mice were weighed (FIG. 1C).

FIGS. 2A-C show that fomepizole and N-acetylcysteine (NAC) do not prevent acetaminophen (AAP) cytotoxicity in vitro. FIG. 2A shows the results of using the Protein Atlas to assess RNA expression of CYP2E1 in the various human organs. FIG. 2B shows the MTT ([3-(4,5-dimethylthiazol-2-yl)-2,5-diphenyltetrazolium bromide]) viability assay results. FIG. 2C shows the results of using the LDH (lactate dehydrogenase) cytotoxicity assay in EF43.fgf4 breast cancer cells. Incubation period 2 days. V=Veh, N=NAC, 0.3 mg/mL, A=AAP, 5 mM, F=fomepizole, 300 μM. T-test, * implies p<0.05. n=3/condition.

FIGS. 3A-C show that fomepizole does not reverse AAP anti-tumor activity in vivo. 4T1 tumors were treated 2×/wk with 650 mg/kg AAP and/or NAC (100 mg/kg) and fomepizole (50 mg/kg) rescue (FIGS. 3A, 3B, 3E; n=5/group) or 500 mg/kg AAP with NAC and propylene glycol (10%) rescue (FIGS. 3C, 3D, 3F; n=3/group). Tumor size and animal weight were measured (FIGS. 3A-D), and BUN/ALT levels were assessed after animals were sacrificed (FIGS. 3E-F).

FIGS. 4A-E show that N-acetylcysteine (NAC) protects against acetaminophen (AAP) associated liver toxicity but not anti-cancer activity. FIG. 4A shows the results of the MTT ([3-(4,5-dimethylthiazol-2-yl)-2,5-diphenyltetrazolium bromide]) viability assay 2 days after treatment with AAP (1 mM), NAC (0.3 mg/mL) and/or propylene glycol (PG, 1%). FIG. 4B shows the histology results of C57bl/6 mice treated with Veh, AAP (500 mg/kg) and/or NAC (100 mg/kg with 10% PG). Mice were sacrificed 24 hrs post treatment. FIG. 4C shows the AAP level measured by ELISA in serum after mouse treated with 500 mg/kg AAP IP. FIG. 4D and FIG. 4E shows the serum levels of BUN and ALT 24 hours after indicated treatment.

FIGS. 5A-C show that administration of acetaminophen (AAP) inhibits expression of M2 but not M1 markers in vitro. FIG. 5A shows bone marrow-derived macrophages (BMDM) were treated for 30 min as indicated with AAP (1 mM) and/or N-acetylcysteine (NAC) (0.3 mg/mL) and Western blot was performed. FIG. 5B and FIG. 5C shows that BMDM were stimulated towards M1 phenotype with LPS and IFN gamma or towards M2 phenotype using IL-4 for 24 hours. BMDM were treated with V (Vehicle), A (AAP, 1 mM) and/or N (NAC, 0.3 mg/mL) overnight and analyzed next day for gene expression using qPCR.

FIGS. 6A-D show that administration of acetaminophen (AAP) increases macrophage phagocytosis. FIG. 6A shows that bone marrow derived macrophages (BMDM) were cultured overnight with EF43.fgf4 breast cancer cells stained with Cellbrite Green along with indicated treatment (Veh, AAP, and/or N-acetylcysteine (NAC)). Cells were analyzed by flow; F4/80 positive cells that were also Cellbrite Green positive were considered phagocytic macrophages and were quantified as a percentage of total cells. FIG. 6B shows the average of three experiments. FIG. 6C shows the representative images. FIG. 6D shows that BMDM were cultured overnight with indicated treatment (Veh, AAP, and/or NAC) and polarized to M0 (no cytokines), M1 (20 ng/mL IFN gamma and 100 ng/mL LPS) or M2 (20 ng/ml IL-4) macrophages overnight. The following day, treatment was removed, and BMDM were placed in serum free media. Cellbrite Green stained LLC-luc tumor cells were added for 4 hours, and fraction of phagocytic macrophages were analyzed using flow cytometry.

FIGS. 7A-E show administration of acetaminophen (AAP) inhibits EF43.fgf4 tumor growth in vivo. FIG. 7A shows that Balb/c mice containing EF43.fgf4 tumors were treated 2×/week with N-acetylcysteine (NAC) (100 mg/kg) or AAP (500 mg/kg) in combination with NAC and that tumor growth was monitored with digital calipers. FIG. 7B shows that tumors were weighed at time of animal sacrifice. FIG. 7C shows that at conclusion of study, tumors were digested and analyzed for IL-10 and IFN gamma by ELISA. FIG. 7D shows that CD45+/CD11B+/F4/80+ macrophages were analyzed for M1 and M2 macrophage markers by flow cytometry. FIG. 7E shows the average MFI of indicated markers in macrophages from 3 mice tumors per treatment group are shown. *≤0.05

FIGS. 8A-B show acetaminophen (AAP) inhibits spheroid formation in NSCLC with diverse oncogenic mutations. FIG. 8A shows NSCLC cells that were plated in CSC media and 4 days later number of spheroids above 50 μM were counted. Results normalized to vehicle. FIG. 7B shows in vitro a limiting dilution assay (LDA) for spheroid formation of AAP (1 mM).

FIGS. 9A-B show acetaminophen (AAP) decreases CSC marker expression in H460 NSCLC cells. Cells in CSC media were treated with Vehicle (Veh), AAP (1 mM) or AAP+N-acetylcysteine (NAC) (1 mg/mL) and analyzed with Western blot (FIG. 9A) and qPCR (FIG. 10B). N=3, * p<0.05.

FIGS. 10A-B show that acetaminophen (AAP) inhibits tumor growth without impairing liver function. FIG. 10A shows NCR/nu mice harboring CD133+H460 NSCLC CSCs treated with vehicle, N-acetylcysteine (NAC) (100 mg/kg), or AAP (350 mg/kg)+NAC. Arrows indicate treatment days. FIG. 10B shows that at time of animal sacrifice, serum was sent for ALT levels. n=5 for Vehicle and AAP+NAC groups, n=4 for NAC group, *<0.05.

FIG. 11 shows that acetaminophen (AAP) inhibits spheroid formation ex vivo. H460 tumors were extracted from nude rats 4 days after in vivo treatment and analyzed for sphere formation in CSC media *<0.05.

FIGS. 12A-C show that the phosphokinase array reveals acetaminophen (AAP) inhibits STAT3. FIG. 12A shows H460 NSCLC cells that were implanted in NCR/nu mice and treated with AAP (500 mg/kg)+/−N-acetylcysteine (NAC) (100 mg/kg). Tumors and livers were extracted and analyzed for glutathione content. FIG. 12B shows H460 cells that were treated with IL-6+/−AAP (10 mM) and analyzed with phosphokinase array. FIG. 12C shows the relative intensity of selected phospho-proteins of AAP-treated cells (relative to vehicle). *<05

FIG. 13 shows acetaminophen (AAP) effects on SRC and ERK. H460 cells were treated with AAP and analyzed with immune-blotting after 2 hr incubation.

FIGS. 14A-C show that acetaminophen (AAP) inhibits STAT3 phosphorylation. H460 (FIG. 14A and FIG. 14B) or CUTO 29 (FIG. 14C) NSCLC cells were grown as spheroids and treated with vehicle, AAP (1 mM)+/−N-acetylcysteine (NAC) (1 mg/mL) for indicated time (FIG. 14A) or 24 hours (FIG. 14B and FIG. 14C).

FIGS. 15A-B show that acetaminophen (AAP) inhibits genes and proteins downstream of STAT3. H460 NSCLC cells were treated with AAP (1 mM) and/or N-acetylcysteine (NAC) (1 mg/mL) for 24 h prior to immunoblotting (FIG. 15A) or pPCR analysis (FIG. 15B). *<0.05 FIGS. 16A-B show acetaminophen (AAP) inhibits CSCs via STAT3. FIG. 16A shows H460 NSCLC cells were transiently transfected with scramble or STAT3 shRNA and treated with vehicle or AAP (1 mM) 4 days in CSC media. Number of spheroids per well were counted.

FIG. 15B shows scramble or STAT3 KD cells were treated with AAP (1 mM) or vehicle and analyzed with immunoblot. Images quantified with ImageJ, normalized to Vehicle and GAPDH. *<0.05

FIGS. 17A-D show that acetaminophen (AAP), unlike C188-9, binds to STAT3 with high specificity relative to STAT1. Spectrofluorimetry experiments were used to determine the binding affinity of AAP and C188-9 to STAT3 (FIG. 17A and FIG. 17 C) and STAT1 (FIG. 17B and FIG. 17D).

FIGS. 18A-C show that acetaminophen (AAP) is a selective STAT3 inhibitor. FIG. 18A and FIG. 18B show that H460 NSCLC cells in CSC media were treated with AAP (1 mM), C188-9 (10 μM), WP1066 (2.5 μM), and napabucasin (Napa) (1 μM) and 4 hours later analyzed by immunoblotting. FIG. 18C shows the semi-quantification of blots shown in FIG. 18A and FIG. 18B using ImageJ software (average of 2 experiments).

FIG. 19 shows Annexin/PI staining. H460 cells in CSC media were treated for 48 hours with cisplatin (Cis, 0.3 μg/mL), and/or acetaminophen (AAP) (1 mM) prior to analysis.

FIGS. 20A-B show Fa-CI plot of cisplatin in combination with acetaminophen (AAP). H460 cells in CSC media were treated with AAP and cisplatin, both separately and together, at increasing concentrations. Spheroids were counted 4 days following treatment. The combination index (CI) was calculated and plotted in a Fa-CI plot (FIG. 20A). FIG. 20B shows CI values at increasing Fa values. Fa represents fraction affected (fraction of spheroids inhibited).

FIGS. 21A-B show BALB/c mice were treated with indicated dose of acetaminophen (AAP)+/−N-acetylcysteine (NAC) (100 mg/kg) and/or fomepizole (30 mg/kg) concurrently. Twenty-four hours later serum and liver histology were analyzed. FIG. 21A shows that NAC is inadequate at preventing AAP toxicity, but fomepizole is. ALT values are shown from serum. FIG. 21B shows mouse was weighed 24 hours after indicated treatment. Mice treated with fomepizole had no weight loss. FIG. 21C shows liver evaluated 24 hours after indicated treatment using H&E stain. Blue arrow shows necrotic area in mice treated with AAP/NAC that was not present if fomepizole was included in rescue cocktail.

### DETAILED DESCRIPTION

The present disclosure can be understood more readily by reference to the following detailed description of the invention, the figures and the examples included herein.

Before the present compositions and methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

Moreover, it is to be understood that unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including matters of logic with respect to arrangement of steps or operational flow, plain meaning derived from grammatical organization or punctuation, and the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

As used in the specification and the appended claims, the singular forms “a,” “an” and “the” include plural referents unless the context clearly dictates otherwise.

The word “or” as used herein means any one member of a particular list and also includes any combination of members of that list.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

Ranges can be expressed herein as from “about” or “approximately” one particular value, and/or to “about” or “approximately” another particular value. When such a range is expressed, a further aspect includes from the one particular value and/or to the other particular value. Similarly, when values are expressed as approximations, by use of the antecedent “about,” or “approximately,” it will be understood that the particular value forms a further aspect. It will be further understood that the endpoints of each of the ranges are significant both in relation to the other endpoint and independently of the other endpoint. It is also understood that there are a number of values disclosed herein and that each value is also herein disclosed as “about” that particular value in addition to the value itself. For example, if the value “10” is disclosed, then “about 10” is also disclosed. It is also understood that each unit between two particular units is also disclosed. For example, if 10 and 15 are disclosed, then 11, 12, 13, and 14 are also disclosed.

As used herein, the terms “optional” or “optionally” mean that the subsequently described event or circumstance may or may not occur and that the description includes instances where said event or circumstance occurs and instances where it does not.

As used herein, the term “subject” refers to the target of administration, e.g., a human. Thus, the subject of the disclosed methods can be a vertebrate, such as a mammal, a fish, a bird, a reptile, or an amphibian. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.). In one aspect, a subject is a mammal. In another aspect, the subject is a human. The term does not denote a particular age or sex. Thus, adult, child, adolescent and newborn subjects, as well as fetuses, whether male or female, are intended to be covered.

As used herein, the term “patient” refers to a subject afflicted with a disease or disorder. The term “patient” includes human and veterinary subjects. In some aspects of the disclosed methods, the “patient” has been diagnosed with a need for treatment for cancer, such as, for example, prior to the administering step.

As used herein, the term “treating” refers to partially or completely alleviating, ameliorating, relieving, delaying onset of, inhibiting or slowing progression of, reducing severity of, and/or reducing incidence of one or more symptoms or features of a particular disease, disorder, and/or condition. Treatment can be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition. For example, the disease, disorder, and/or condition can be cancer. In some aspects, the disease, disorder, and/or condition can be pain and/or fever.

As used herein, the term “inhibit” or “inhibiting” mean decreasing tumor cell growth rate from the rate that would occur without treatment and/or causing tumor mass (e.g., cancer) to decrease. Inhibiting also include causing a complete regression of the tumor (e.g., cancer).

“Inhibit,” “inhibiting” and “inhibition” also mean to diminish or decrease an activity, response, condition, disease, or other biological parameter. This can include, but is not limited to, the complete ablation of the activity, response, condition, or disease. This may also include, for example, a 10% inhibition or reduction in the activity, response, condition, or disease as compared to the native or control level. Thus, in an aspect, the inhibition or reduction can be a 10, 20, 30, 40, 50, 60, 70, 80, 90, 100%, or any amount of reduction in between as compared to native or control levels. In some aspects, the inhibition or reduction is 10-20, 20-30, 30-40, 40-50, 50-60, 60-70, 70-80, 80-90, or 90-100% as compared to native or control levels. In an aspect, the inhibition or reduction is 0-25, 25-50, 50-75, or 75-100% as compared to native or control levels.

The terms “reducing,” “inhibiting” and “ameliorating,” as used herein, when used in the context of modulating a pathological or disease state, generally refers to the prevention and/or reduction of at least a portion of the negative consequences of the disease state. When used in the context of an adverse side effect associated with the administration of a drug to a subject, the term(s) generally refer to a net reduction in the severity or seriousness of said adverse side effects.

The term “acetaminophen analog” refers to any chemical derivative of acetaminophen. Examples are listed in Fresno et al. (2014) Adamantyl Analogues of Paracetamol as Potent Analgesic Drugs via Inhibition of TRPA1. PLOS ONE 9 (12): e113841; Nilsson et al. (2021) Paracetamil analogues conjugated by FAAH induce TRPV1-meidated antinociception without causing acute liver toxicity. European Journal of Medicinal Chemistry 213:113042; Nam et al. (2009) Pyridine and pyrimidine analogs of acetaminophen as inhibitors of lipid peroxidation and cyclooxygenase and lipoxygenase catalysis. Org Biomol Chem 7 (24): 5103-5112; Bazan et al. (2020) A novel pipeline of 2-(benzenesulfonamide)-N-(4-hydroxyphenyl) acetaminde analgesics that lack hepatotoxicity and retain antipyresis. European Journal of Medicinal Chemistry 2020: 112600; U.S. Publication No. 2020-0002364; and U.S. Pat. No. 11,021,498, which are hereby incorporated in their entirety for their teaching of acetaminophen analogs.

As disclosed herein, it was tested whether the anti-tumor effect of high-dose AAP was mediated by a mechanism similar to the known hepatocellular injury effects of AAP overdose. Within the liver, AAP gets metabolized by the mixed function oxidase (MFO) family of enzymes (specifically CYP2E1), and overdose leads to a buildup of a toxic free radical metabolite NAPQI. NAPQI gets detoxified by the anti-oxidant glutathione, leading to glutathione depletion and reactive oxygen species mediated hepatocellular injury (Heard K J. N Engl J Med. 2008; 359 (3): 285-92). However, accumulating pre-clinical and clinical data suggest that the anti-cancer effects of AAP are ROS-independent. A reactive oxygen species (ROS) independent mechanism of cytotoxicity was supported by a phase I study of AAP in patients with metastatic melanoma in which no significant glutathione depletion was noted in tumors or peripheral blood monocytic cells (PBMCs) of AAP-treated patients (Wolchok J D, et al. Melanoma Res. 2003; 13 (2): 189-96). These findings are consistent with in vivo animal studies of tumor-bearing rats, which demonstrated glutathione depletion within the liver but not in the tumor (Wu Y J, et al. Anticancer Res. 2013; 33 (6): 2391-400) The selective glutathione depletion within the liver is likely secondary to increased CYP2E1 expression in hepatocytes relative to other organs (Bieche I, et al. Pharmacogenet Genomics. 2007; 17 (9): 731-42). Given the findings that AAP selectively depletes glutathione within the liver and not the tumor in vivo, NAC (a glutathione precursor) may selectively rescue the liver but not the tumor from high dose AAP treatment. However, if the anti-tumor activity of high dose AAP is not mediated by glutathione depletion, then an alternate mechanism exists.

Disclosed herein are data describing a free-radical independent mechanistic explanation for the anti-tumor effect of high dose AAP. It was tested whether high dose AAP acts as an inhibitor of STAT3 (via direct binding) and CSCs, an effect not reversed by concurrent administration of NAC. These findings are of high clinical translational relevance and provide the mechanistic basis for a therapeutic approach for treating NSCLC with high-dose AAP administered concurrently with NAC for the selective rescue of the normal liver.

Most pre-clinical studies of therapeutic approaches that show promise in mouse models fail to be effective in human clinical trials. The clinical study of high dose AAP has not progressed beyond phase I despite promising evidence of anti-tumor activity against multiple histologies [3/14 (21%) assessable patients with objective responses in the initial trial (Kobrinsky N L, et al. Cancer Invest. 1996; 14 (3): 202-10)]. Drugs with a 20% response rate or higher in phase I (less than 10% of drugs evaluated in phase I) have a 51% chance of meeting primary endpoints in subsequent phase II trials (Bugano D D G, et al. Clin Cancer Res. 2017; 23 (15): 4020-6). The lack of further clinical evaluation is likely due in part to the impracticality of administering 20 g/m² of AAP orally (about 40 grams, or 80 extra strength acetaminophen tablets), leading to extreme nausea. However, an IV AAP formulation has since become FDA-approved (Ofirmev®). Further, lack of mechanistic understanding of high dose AAP's anti-tumor activity has also likely contributed to the failure of high dose AAP to be developed as an anti-cancer therapeutic.

Disclosed herein is a CSC inhibitor, high dose AAP with NAC rescue, in the pre-clinical management of lung cancer. Further disclosed herein are compositions comprising high-dose AAP combined with NAC that can be further combined with chemotherapies including but not limited to cisplatin, and tyrosine kinase inhibitor (TKI) therapy in driver mutation-positive NSCLC, in order to help veterans with metastatic lung cancer achieve durable remissions.

Further disclosed herein are formulations of high dose AAP with NAC and fomepizole rescue for treating cancer. Also disclosed herein are formulations of high dose AAP combined with NAC and propylene glycol (PG) rescue for treating cancer.

Acetaminophen is metabolized by a few pathways; the CYP2E1 metabolism pathway generates a toxic free radical (NAPQI) that results in glutathione depletion and free radical injury. The CYP2E1 pathway is a minor metabolic pathway, responsible for 10% of AAP metabolism. The CYP2E1 pathway is unlikely to be responsible for AAP therapeutic benefits in pain or cancer, in large part, because NAPQI is a transient free radical that is detoxified locally by glutathione in the liver; thus, it is unlikely that it has systemic therapeutic benefits. CYP2E1 is expressed exclusively in the liver, and is not generated systemically.

It has been shown that high dose AAP results in glutathione depletion in the liver but not the tumor; these results show that NAC, a glutathione precursor, may selectively rescue the liver but not the tumor from high dose AAP treatment. Additionally, these results suggest differential mechanisms of anti-cancer activity of high dose AAP from the toxicity mechanism. The differential effects of AAP on liver and elsewhere is likely because CYP2E1 is selectively expressed in the liver.

It has also been shown that high dose AAP functions as a STAT3 inhibitor, an effect that is not reversed by concurrent treatment with NAC. Thus, a free radical independent mechanism of anti-cancer activity has been established that further supports the concept that AAP toxicity may be selectively rescued with NAC (and potentially other antidotes) without compromising anti-cancer activity.

The CYP2E1 pathway is responsible for liver toxicity. It has been shown that CYP2E1 KO mice are resistant to AAP toxicity.

NAC alone is a poor antidote to AAP toxicity when given concurrently because it takes time for CYP2E1 to metabolize AAP into free radicals and for glutathione to be depleted. By the time glutathione is depleted, NAC has already been metabolized. Clinically, to circumvent this issue, NAC is given as a long infusion; practically speaking long IV NAC infusions may not be convenient in most circumstances.

Fomepizole and propylene glycol (PG) are both CYP2E1 inhibitors. Concurrent timing of CYP2E1 inhibitor treatment with AAP is appropriate because, unlike NAC, which is effective once free radicals have been generated, CYP2E1 inhibition prevents the formation of free radicals by AAP metabolism. Thus, fomepizole and propylene glycol can be given concurrently with AAP to prevent toxicity without compromising the therapeutic benefits of AAP.

As disclosed herein, rescue cocktail of NAC plus fomepizole can be administered up to 650 mg/kg AAP (100-fold higher than therapeutic doses) to mice without any toxicity (e.g., ALT, BUN, liver histology, weight changes). Additionally, fomepizole does not interfere with anti-cancer activity of high dose AAP in vivo and is unlikely to interfere with its pain and other effects as well. This finding is because 90% of acetaminophen is metabolized by other pathways that are relatively unaffected by CYP2E1 inhibition. Thus, blocking CYP2E1 inhibits the toxicity metabolic pathway of AAP; however, the remaining pathways that are involved in therapeutic benefit (e.g., the AAP metabolite AM 404 involved in pain relief) are unaffected.

Further described herein is a cocktail of propylene glycol plus NAC is effective at preventing toxicity in the liver. For example, the results described herein demonstrate that zone 3 necrosis in the AAP treated liver is not present in the mouse that was treated with concurrent NAC+PG rescue. Additionally, mice treated with a combination of AAP, NAC, and PG have normal serum BUN and ALT values, demonstrating no liver or kidney toxicity.

Disclosed herein are compositions and methods comprising fomepizole and/or propylene glycol administration concurrent with AAP administration can be used to allow safe dose escalation of AAP for enhanced therapeutic benefit of cancer and pain without toxicity.

### Compositions

Disclosed herein are composition comprising: acetaminophen or an analog thereof; and a CYP2E1 inhibitor. In some aspects, the compositions can further comprise a pharmaceutical acceptable carrier.

Disclosed herein are pharmaceutical liquid compositions. In some aspects, the pharmaceutical liquid compositions can comprise: acetaminophen or an analog thereof, and a CYP2E1 inhibitor. In some aspects, the pharmaceutical liquid composition can further comprise a pharmaceutical acceptable carrier. In some aspects, the carrier can be saline.

In some aspects, the CYP2E1 inhibitor can be fomepizole, propylene glycol, disulfiram, indazole, diallyl sulfide, clotrimazole, or isoniazide.

In some aspects, the acetaminophen analog thereof can be Kp-1199, N-[2-(3,4-dihydroxyphenil) ethyl]-2-[[2-(4-hydroxyanilino)-2-oxo-ethyl]sulfamoyl] bensamide, 2-[[2-(40hydroxyanilino)-2-oxo-ethyl]sulfamoyl]-N-methylbenzamide, or 5-amino-2-methoxyphenol, 5-aminoindazole. In some aspects, the acetaminophen or analog thereof can be one or more of the compounds or structures listed below:
(4-acetamidophenoxy)methyl dihydrogen phosphate;
Sodium (4-acetamidophenoxy)methyl phosphate;
(4-acetamidophenoxy)methyl dihydrogen phosphate N-(4-(methylthiomethoxy)phenyl) acetamide;
(4-acetamidophenoxy)methyl dibenzyl phosphate;

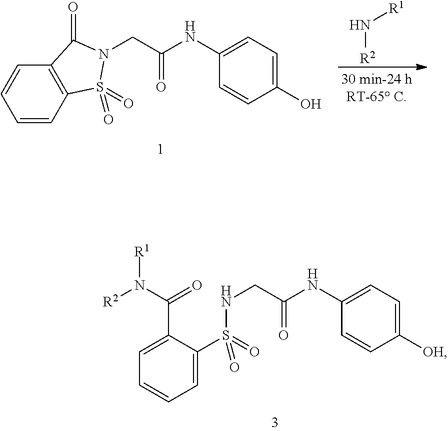

wherein R¹ and R² can be

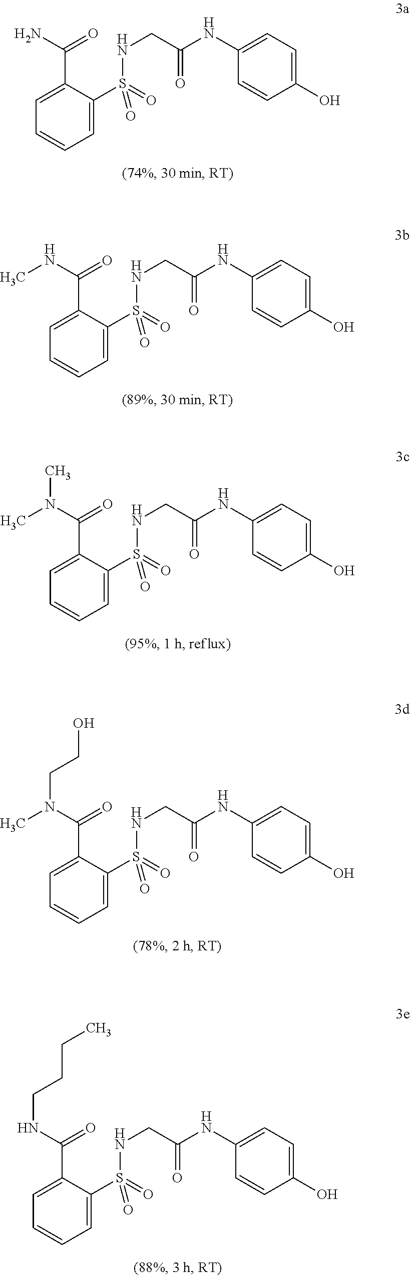 [SMILES: NC(=O)c1ccccc1S(=O)(=O)NCC(=O)Nc1ccc(O)cc1 ; CNC(=O)c1ccccc1S(=O)(=O)NCC(=O)Nc1ccc(O)cc1 ; CN(C)C(=O)c1ccccc1S(=O)(=O)NCC(=O)Nc1ccc(O)cc1 ; CN(CCO)C(=O)c1ccccc1S(=O)(=O)NCC(=O)Nc1ccc(O)cc1 ; CCCCNC(=O)c1ccccc1S(=O)(=O)NCC(=O)Nc1ccc(O)cc1]

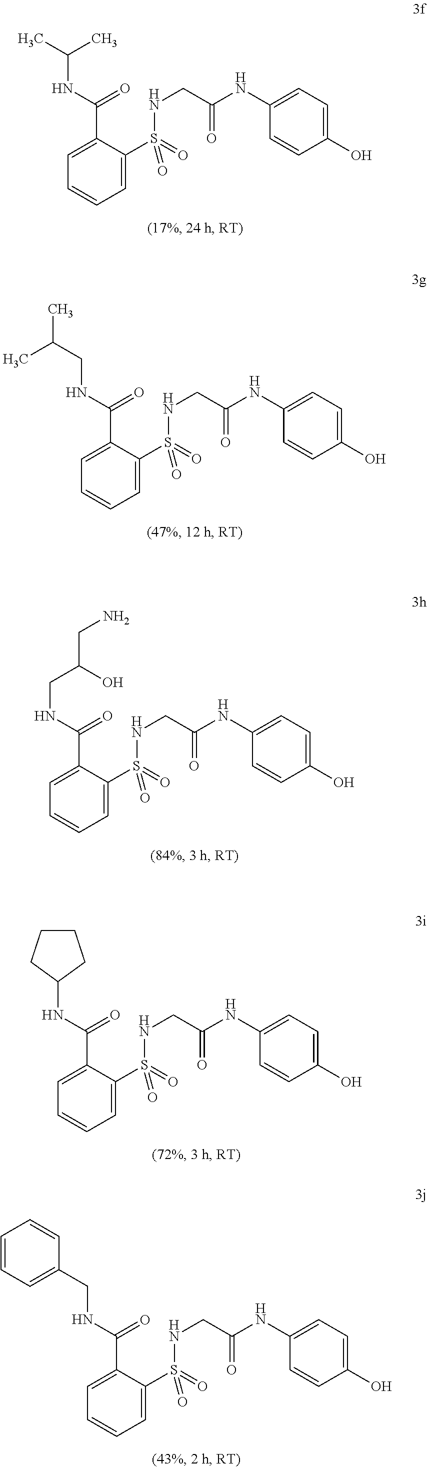 [SMILES: CC(C)NC(=O)c1ccccc1S(=O)(=O)NCC(=O)Nc1ccc(O)cc1 ; CC(C)CNC(=O)c1ccccc1S(=O)(=O)NCC(=O)Nc1ccc(O)cc1 ; NCC(O)CNC(=O)c1ccccc1S(=O)(=O)NCC(=O)Nc1ccc(O)cc1 ; O=C(CNS(=O)(=O)c1ccccc1C(=O)NC1CCCC1)Nc1ccc(O)cc1 ; O=C(CNS(=O)(=O)c1ccccc1C(=O)NCc1ccccc1)Nc1ccc(O)cc1]

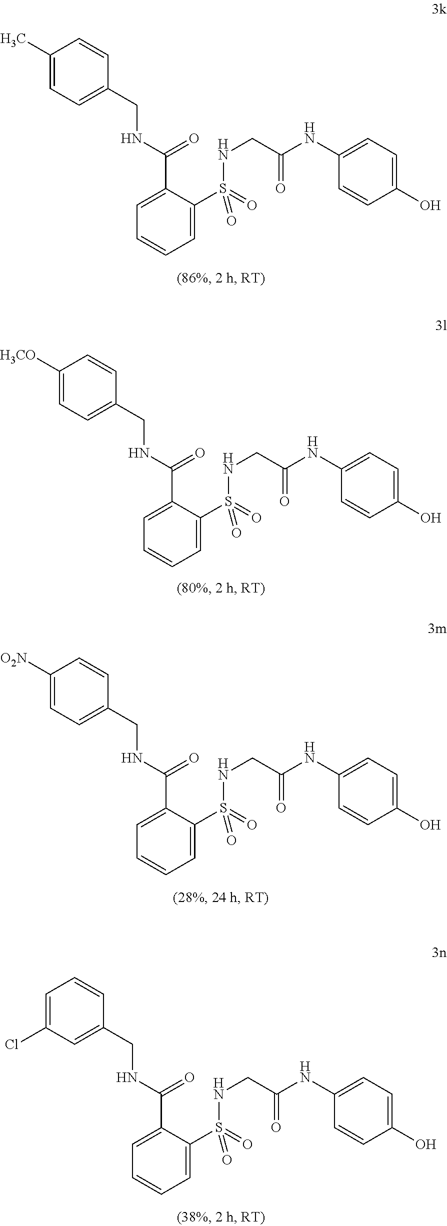 [SMILES: Cc1ccc(CNC(=O)c2ccccc2S(=O)(=O)NCC(=O)Nc2ccc(O)cc2)cc1 ; COc1ccc(CNC(=O)c2ccccc2S(=O)(=O)NCC(=O)Nc2ccc(O)cc2)cc1 ; O=C(CNS(=O)(=O)c1ccccc1C(=O)NCc1ccc([N+](=O)[O-])cc1)Nc1ccc(O)cc1 ; O=C(CNS(=O)(=O)c1ccccc1C(=O)NCc1cccc(Cl)c1)Nc1ccc(O)cc1]

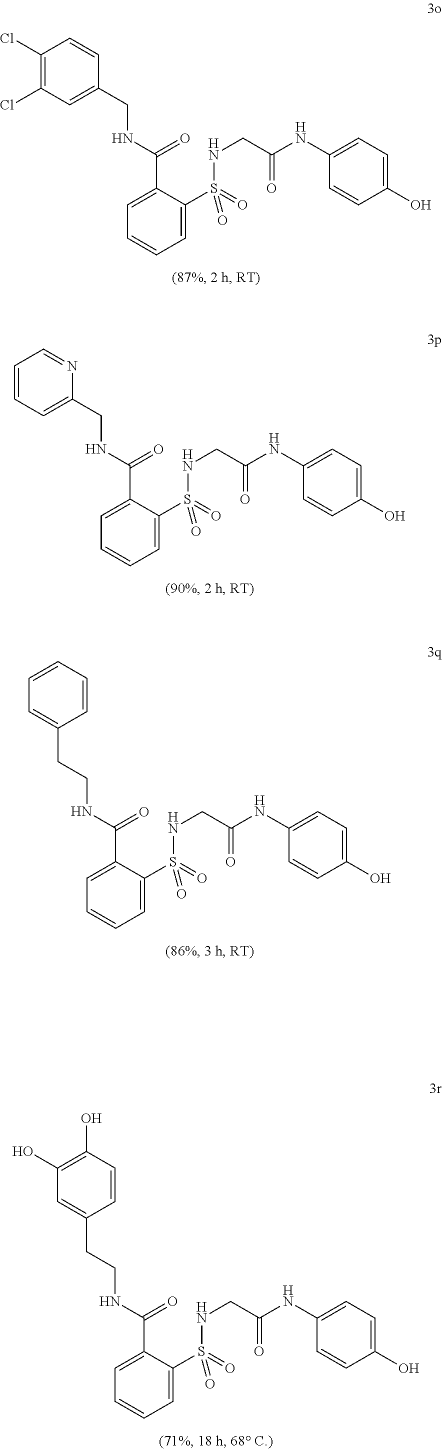 [SMILES: O=C(CNS(=O)(=O)c1ccccc1C(=O)NCc1ccc(Cl)c(Cl)c1)Nc1ccc(O)cc1 ; O=C(CNS(=O)(=O)c1ccccc1C(=O)NCc1ccccn1)Nc1ccc(O)cc1 ; O=C(CNS(=O)(=O)c1ccccc1C(=O)NCCc1ccccc1)Nc1ccc(O)cc1 ; O=C(CNS(=O)(=O)c1ccccc1C(=O)NCCc1ccc(O)c(O)c1)Nc1ccc(O)cc1]

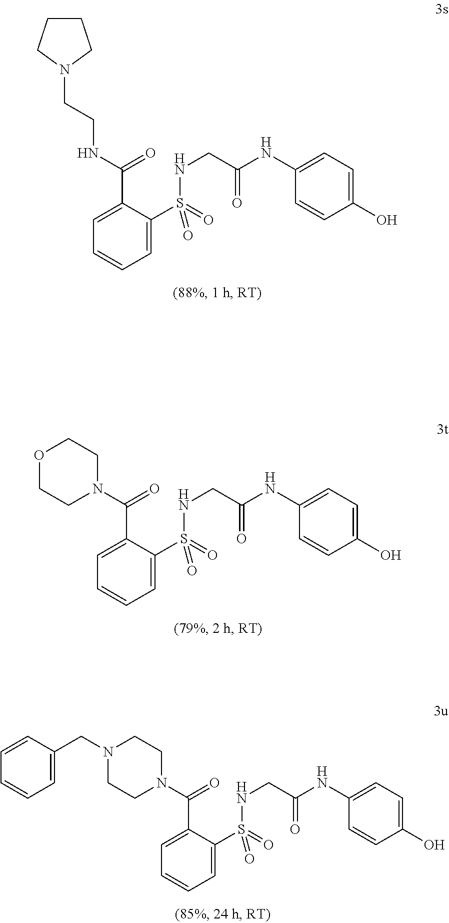 [SMILES: O=C(CNS(=O)(=O)c1ccccc1C(=O)NCCN1CCCC1)Nc1ccc(O)cc1 ; O=C(CNS(=O)(=O)c1ccccc1C(=O)N1CCOCC1)Nc1ccc(O)cc1 ; O=C(CNS(=O)(=O)c1ccccc1C(=O)N1CCN(Cc2ccccc2)CC1)Nc1ccc(O)cc1]

hydroxyanilino)-2-oxo-ethyl]sulfamoyl]benzamide;
3b: 2-[[2-(4-Hydroxyanilino)-2-oxo-ethyl]sulfamoyl]-N-methylbenzamide;
3c: 2-[[2-(4-Hydroxyanilino)-2-oxo-ethyl]sulfamoyl]-N,N-dimethylbenzamide;
3d: 2-[[2-(4-Hydroxyanilino)-2-oxo-ethyl]sulfamoyl]-N-(2-hydroxyethyl)-N-methyl-benzamide;
3e: N-Butyl-2-[[2-(4-hydroxyanilino)-2-oxo-ethyl]sulfamoyl]benzamide;
3f: 2-[[2-(4-Hydroxyanilino)-2-oxo-ethyl]sulfamoyl]-N-isopropylbenzamide;
3g: 2-[[2-(4-Hydroxyanilino)-2-oxo-ethyl]sulfamoyl]-N-isobutylbenzamide;
3h: N-(3-Amino-2-hydroxypropyl)-2-[[2-(4-hydroxyanilino)-2-oxo-ethyl]sulfamoyl]benzamide;
3i: N-Cyclopentyl-2-[[2-(4-hydroxyanilino)-2-oxo-ethyl]sulfamoyl]benzamide;
3j: N-Benzyl-2-[[2-(4-hydroxyanilino)-2-oxo-ethyl]sulfamoyl]benzamide;
3k: 2-[[2-(4-Hydroxyanilino)-2-oxo-ethyl]sulfamoyl]-N-[(4-methylphenyl)methyl]benzamide;
3l: 2-[[2-(4-Hydroxyanilino)-2-oxo-ethyl]sulfamoyl]-N-[(4-methoxyphenyl)methyl]benzamide;
3m: 2-[[2-(4-Hydroxyanilino)-2-oxo-ethyl]sulfamoyl]-N-[(4-nitrophenyl)methyl]benzamide;
3n: N-[(3-Chlorophenyl)methyl]-2-[[2-(4-hydroxyanilino)-2-oxo-ethyl]sulfamoyl]benzamide;
3o: N-[(3,4-Dichlorophenyl)methyl]-2-[[2-(4-hydroxyanilino)-2-oxo-ethyl]sulfamoyl]benzamide;
3p: 2-[[2-(4-Hydroxyanilino)-2-oxo-ethyl]sulfamoyl]-N-(2-pyridylmethyl)benzamide;
3q: 2-[[2-(4-Hydroxyanilino)-2-oxo-ethyl]sulfamoyl]-N-(2-phenylethyl)benzamide;
3r: N-[2-(3,4-Dihydroxyphenyl)ethyl]-2-[[2-(4-hydroxyanilino)-2-oxo-ethyl]sulfamoyl]benzamide;
3s: 2-[[2-(4-Hydroxyanilino)-2-oxo-ethyl]sulfamoyl]-N-(2-pyrrolidin-1-ylethyl)benzamide;
3t: N-(4-Hydroxyphenyl)-2-[[2-(morpholine-4-carbonyl)phenyl]sulfonylamino]acetamide 3;
3u: 2-[[2-(4-Benzylpiperazine-1-carbonyl)phenyl]sulfonylamino]-N-(4-hydroxyphenyl) acetamide;

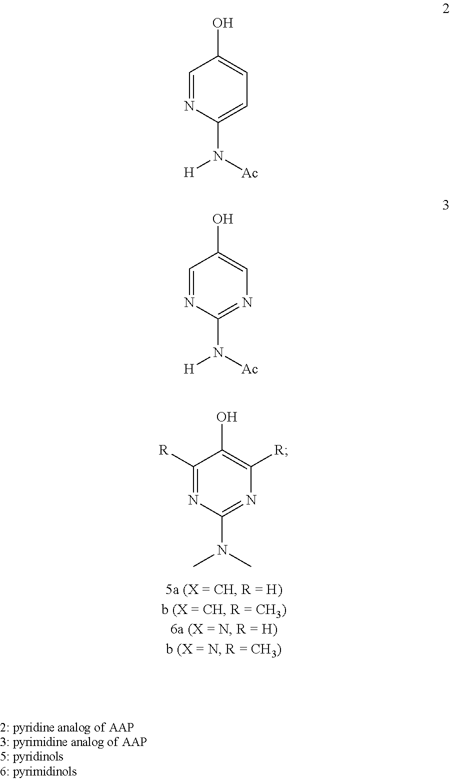

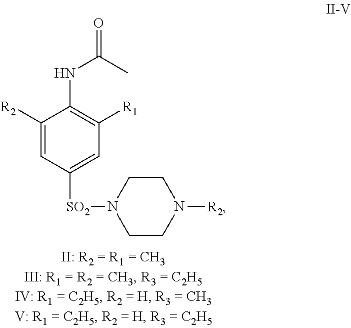

II. N-[4-(Methylpiperazine-4-ylsulfonyl)-2,6-dimethylphenyl]acetamide (II),
III: N-[4-(ethylpiperazin-4-ylsulfonyl)-2,6-dimethylphenyl]acetamide (III),
IV: N-[4-(methylpiperazin-4-ylsulfonyl)-2-ethylphenyl]acetamide
V: N-[4-(ethylpiperazin-4-ylsulfonyl)-2-ethylphenyl]acetamide;

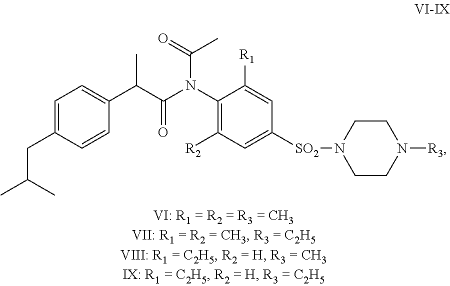

VI: N-acetyl-N-{2,6-dimethyl-4-[(4-methylpiperazin-1-yl)-sulfonyl]phenyl}-2-(4-isobutylphenyl) propanamide,
VII: N-acetyl-N-{4-[(4-ethylpiperazin-1-yl) sulfonyl]-2,6-dimethylphenyl}-2-(4-isobutylphenyl) propanamide,
VIII: N-acetyl-N-{2-ethyl-4-[(4-methylpiperazin-1-yl) sulfonyl]-phenyl}-2-(4-isobutylphenyl) propanamide
IX: N-acetyl-N-{2-ethyl-4-[(4-ethylpiperazin-1-yl) sulfonyl]-phenyl}-2-(4-isobutylphenyl) propanamide (IX).

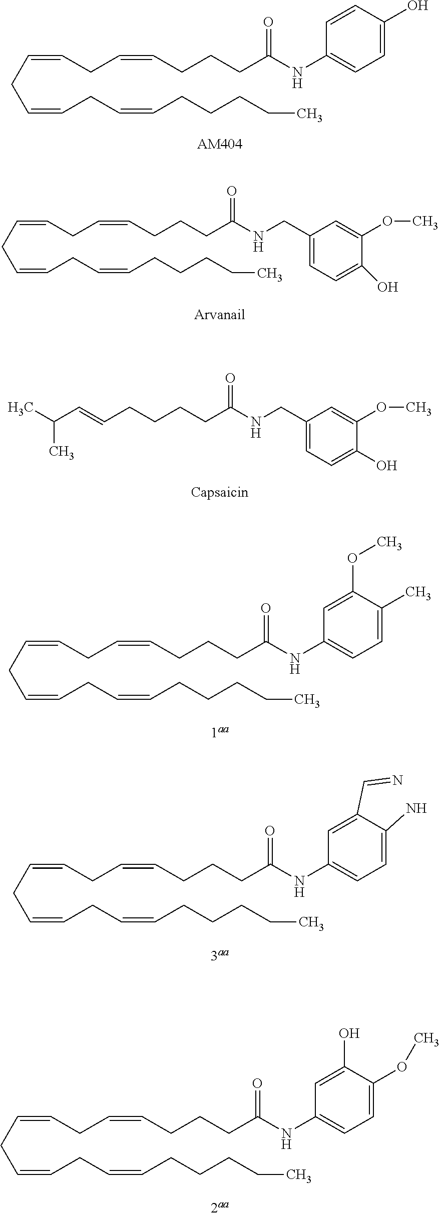

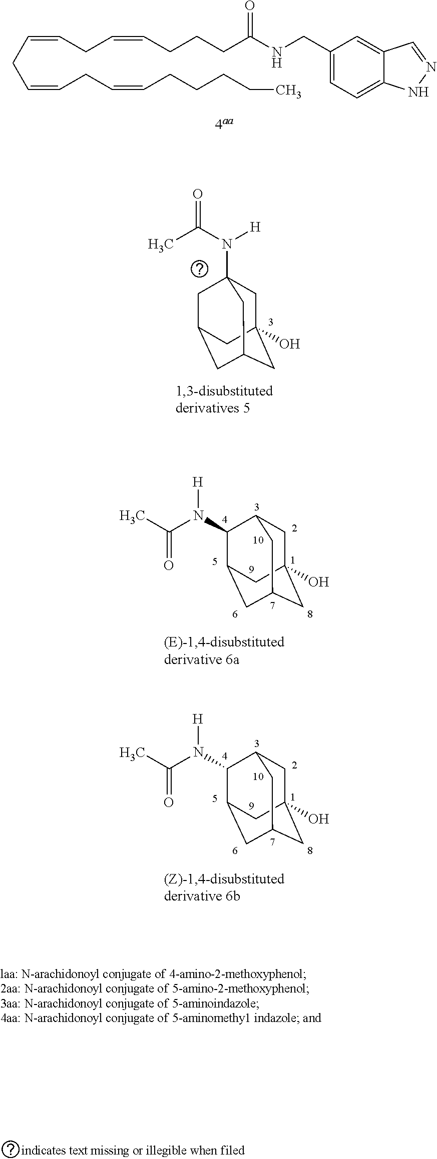

In some aspects, the composition can further comprise an antioxidant. In some aspects, the antioxidant can be N-acetylcysteine, sodium thiosulfate, or amifostine. In some aspects, the antioxidant is not N-acetylcysteine.

In some aspects, a “standard dose” of acetaminophen is about 325 mg to 650 mg every 4 to 6 hours. In some aspects, about 650 mg up to 4 times/day is considered a “high dose” of acetaminophen. In some aspects, “high dose” of acetaminophen for treating pain is up to 1 g every 6 hours. In some aspects, the maximum daily dose of acetaminophen is 4 g/day.

In some aspects, the acetaminophen or the analog thereof can be present in amount of at least 1-200 g. In some aspects, the acetaminophen or the analog thereof can be present in amount of at least 1-400 g. In some aspects, the acetaminophen or the analog thereof can be present in amount of at least 1-200 g administered over a period of time. In some aspects, the acetaminophen or the analog thereof can be present in amount of at least 1-400 g administered over a period of time. In some aspects, the acetaminophen or the analog thereof can be present in amount of at least 1-200 g administered over 1 hour, 2 hours, 3 hours, 4 hours, 5 hours, 6 hours, 7 hours, 8, hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, or 24 hours or any period of time in between. In some aspects, the acetaminophen or the analog thereof can be present in amount of at least 1-400 g administered over 1 hour, 2 hours, 3 hours, 4 hours, 5 hours, 6 hours, 7 hours, 8, hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, or 24 hours or any period of time in between.

In some aspects, the acetaminophen or the analog thereof can be present in amount of at least 0.1-200 g. In some aspects, the acetaminophen or the analog thereof can be present in amount of at least 0.1-400 g. In some aspects, the acetaminophen or the analog thereof can be present in amount of at least 0.1-200 g administered over a period of time. In some aspects, the acetaminophen or the analog thereof can be present in amount of at least 0.1-400 g administered over a period of time. In some aspects, the acetaminophen or the analog thereof can be present in amount of at least 0.1-400 g administered over 1 hour, 2 hours, 3 hours, 4 hours, 5 hours, 6 hours, 7 hours, 8, hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, or 24 hours or any period of time in between.

In some aspects, about 10 mL to 10 L of the composition or liquid pharmaceutical composition can provide a therapeutically effective dose.

In some aspects, about 0.1 mL to 10 L of the composition or liquid pharmaceutical composition can provide a therapeutically effective dose. In some aspects, about 0.1 mL to 1 mL, 1 mL to 10 mL, 10 mL to 20 mL, 20 mL to 30 mL, 30 mL to 40 mL, 40 mL to 50 mL, 50 mL to 60 mL, 60 mL to 70 mL, 70 mL to 80 mL, 80 mL to 90 mL, 90 mL to 100 mL, 100 mL to 200 mL, 200 mL to 300 mL, 300 mL to 400 mL, 400 mL to 500 mL, 500 mL to 600 mL, 600 mL to 700 mL, 700 mL to 800 mL, 800 mL to 900 mL, 900 mL to 1 L, 1 L to 2 L, 2 L to 3 L, 3 L to 4 L, 4 L to 5 L, 5 L to 6 L, 6 L to 7 L, 7 L to 8 L, 8 L to 9 L, or 9 L to 10 L of the composition or liquid pharmaceutical composition can provide a therapeutically effective dose.

In some aspects, the composition or pharmaceutical composition can be in an aqueous form.

In some aspects, the pH of the pharmaceutical liquid composition can range from 3 to 11. In some aspects, the pH of the pharmaceutical liquid composition can ranges from 6 to 8.

In some aspects, the pharmaceutical liquid composition can further comprise an excipient and purified water. In some aspects, the excipient can be one or more of a pH adjuster, a stabilizer, a preservative, a sweetener, and a fragrance ingredient. In some aspects, the pH adjuster can be an alkalizing agent. In some aspects, the alkalizing agent can be one or more of sodium hydroxide, potassium hydroxide, sodium bicarbonate, ammonia solution, potassium citrate, triethanolamine, and sodium citrate.

### Pharmaceutical Compositions

As disclosed herein, are pharmaceutical compositions, comprising one or more of the therapeutic compositions or inhibitors disclosed herein. In some aspects, the compositions can be formulated for oral or parental administration. In some aspects, the parental administration can be intravenous, subcutaneous, intramuscular or direct injection. The compositions can be formulated for administration by any of a variety of routes of administration, and can include one or more physiologically acceptable excipients, which can vary depending on the route of administration. As used herein, the term “excipient” means any compound or substance, including those that can also be referred to as “carriers” or “diluents.” Preparing pharmaceutical and physiologically acceptable compositions is considered routine in the art, and thus, one of ordinary skill in the art can consult numerous authorities for guidance if needed.

The compositions can be administered directly to a subject. Generally, the compositions can be suspended in a pharmaceutically acceptable carrier (e.g., physiological saline or a buffered saline solution) to facilitate their delivery. Encapsulation of the compositions in a suitable delivery vehicle (e.g., polymeric microparticles or implantable devices) may increase the efficiency of delivery.

The compositions described herein can be administered to the subject (e.g., a human patient) in an amount sufficient to delay, reduce, or preferably prevent the onset of clinical disease. Accordingly, in some aspects, the patient can be a human patient. In therapeutic applications, compositions are administered to a subject (e.g., a human patient) already with or diagnosed with cancer (or pain or fever) in an amount sufficient to at least partially improve a sign or symptom or to inhibit the progression of (and preferably arrest) the symptoms of the condition, its complications, and consequences. An amount adequate to accomplish this is defined as a “therapeutically effective amount.” A therapeutically effective amount of a composition (e.g., a pharmaceutical composition) can be an amount that achieves a cure, but that outcome is only one among several that can be achieved. As noted, a therapeutically effective amount includes amounts that provide a treatment in which the onset or progression of the cancer is delayed, hindered, or prevented, or the cancer or a symptom of the cancer is ameliorated. In some aspects, a therapeutically effective amount includes amounts that provide a treatment in which the onset or progression of the pain or fever is delayed, hindered, or prevented, or the pain or a symptom of the pain is ameliorated. One or more of the symptoms can be less severe. Recovery can be accelerated in an individual who has been treated.

Therapeutic administration encompasses prophylactic applications. Based on genetic testing and other prognostic methods, a physician in consultation with their patient can choose a prophylactic administration where the patient has a clinically determined predisposition or increased susceptibility (in some cases, a greatly increased susceptibility) to a type of cancer.

The compositions can be formulated in various ways for parenteral or nonparenteral administration. Where suitable, oral formulations can take the form of tablets, pills, capsules, or powders, which may be enterically coated or otherwise protected. Sustained release formulations, suspensions, elixirs, aerosols, and the like can also be used.

Pharmaceutically acceptable carriers and excipients can be incorporated (e.g., water, saline, aqueous dextrose, and glycols, oils (including those of petroleum, animal, vegetable or synthetic origin), starch, cellulose, talc, glucose, lactose, sucrose, gelatin, malt, rice, flour, chalk, silica gel, magnesium stearate, sodium stearate, glycerol monosterate, sodium chloride, dried skim milk, glycerol, propylene glycol, ethanol, and the like). The compositions may be subjected to conventional pharmaceutical expedients such as sterilization and may contain conventional pharmaceutical additives such as preservatives, stabilizing agents, wetting or emulsifying agents, salts for adjusting osmotic pressure, buffers, and the like. Suitable pharmaceutical carriers and their formulations are described in “Remington's Pharmaceutical Sciences” by E. W. Martin, which is herein incorporated by reference. Such compositions will, in any event, contain an effective amount of the compositions together with a suitable amount of carrier so as to prepare the proper dosage form for proper administration to the patient.

The pharmaceutical compositions as disclosed herein can be prepared for oral or parenteral administration. Pharmaceutical compositions prepared for parenteral administration include those prepared for intravenous (or intra-arterial), intramuscular, subcutaneous, intraperitoneal, transmucosal (e.g., intranasal, intravaginal, or rectal), or transdermal (e.g., topical) administration. Aerosol inhalation can also be used. Thus, compositions can be prepared for parenteral administration that includes acetaminophen or an analog thereof and a CYP2E1 inhibitor dissolved or suspended in an acceptable carrier, including but not limited to an aqueous carrier, such as water, buffered water, saline, buffered saline (e.g., PBS), and the like. One or more of the excipients included can help approximate physiological conditions, such as pH adjusting and buffering agents, tonicity adjusting agents, wetting agents, detergents, and the like. Where the compositions include a solid component (as they may for oral administration), one or more of the excipients can act as a binder or filler (e.g., for the formulation of a tablet, a capsule, and the like).

The pharmaceutical compositions can be sterile and sterilized by conventional sterilization techniques or sterile filtered. Aqueous solutions can be packaged for use as is, or lyophilized, the lyophilized preparation, which is encompassed by the present disclosure, can be combined with a sterile aqueous carrier prior to administration. The pH of the pharmaceutical compositions typically will be between 3 and 11 (e.g., between about 5 and 9) or between 6 and 8 (e.g., between about 7 and 8). In some aspects, the formulation (e.g., formulations amenable to parenteral administration) is an aqueous formulation with a pH from about 3.5 to about 9.5, or from about 4.5 to about 8.5, or from about 5.0 to about 9.0, or from about 5.5 to about 8.5, or from about 6.0 to about 8.0, or from about 6.5 to about 8.0, or from about 7.0 to about 8.0, or about 7.4.

The resulting compositions in solid form can be packaged in multiple single dose units, each containing a fixed amount of the above-mentioned agent or agents, such as in a sealed package of tablets or capsules.

In some aspects, the formulations disclosed herein can vary or be tailored according to the disease, disorder, or condition or the severity of the disease, disorder, or condition to be treated, the amount of compound to be administered, the condition of the individual, and other variables that will readily be apparent to one of ordinary skill in the art in view of the teachings provided herein.

### Methods of Treatment

Disclosed herein are methods of reducing or inhibiting growth of a tumor in a subject. In some aspects, the methods can comprise administering to the subject a therapeutically effective amount of any of the compositions or the pharmaceutical compositions disclosed herein. For example, disclosed herein are methods reducing or inhibiting growth of a tumor in a subject, the methods can comprise administering to the subject a therapeutically effective amount of the composition comprising: acetaminophen or an analog thereof; and CYP2E1 inhibitor or the pharmaceutical composition comprising: acetaminophen or an analog thereof, and CYP2E1 inhibitor. Further, disclosed herein are methods of reducing or inhibiting growth of a tumor in a subject, the methods can comprise administering to the subject a therapeutically effective amount of acetaminophen or an analog thereof and a therapeutically effective amount of a CYP2E1 inhibitor. In some aspects, the therapeutically effective amount of acetaminophen can be administered to the subject, concurrently, simultaneously, or sequentially with the therapeutically effective amount of a CYP2E1 inhibitor. In some aspects, the size of the tumor or the level of one or more tumor markers can be reduced by at least 10%. In some aspects, the growth of the size of the tumor can be less than 10% after the administration of the composition or the pharmaceutical composition disclosed herein compared to the growth of the size of the tumor before administration of the composition or the pharmaceutical composition disclosed herein. In some aspects, the methods can reduce the growth rate of the level of one or more tumor markers by at least 10% after the administration of the composition or the pharmaceutical composition disclosed herein compared to the growth rate of the one or more tumor markers before administration of the composition or the pharmaceutical composition disclosed herein.

In some aspects, the disclosed compositions are capable of reducing, or reduces the size of the tumor or the level of one or more tumor markers by at least 5%, 10%, 15%, 20%, 21%, 22%, 23%, 24%, 25%, 26%, 27%, 28%, 29%, 30%, or more relative to a composition (e.g., a vehicle control) or relative to the size of the tumor or the level of one or more tumor markers prior to administering the compositions disclosed herein to the subject. In some aspects, any of one of the tumor markers described herein can be detected using an antibody-based detection assay (e.g., ELISA), molecular amplification assay (e.g., PCR) or a suitable blood-based assay.

In some aspects, the tumor marker can be anything present in or produced by cancer cells or other cells of the body in response to cancer or certain benign (noncancerous) conditions that provides information about a cancer, including but not limited to how aggressive it is, what kind of treatment it may respond to, or whether it is responding to treatment. Examples of tumor markers include but are not limited to CEA, CA 19-9, alpha fetoprotein, and lactate dehydrogenase.

Disclosed herein are methods of inducing apoptosis of a cancer cell. In some aspects, the methods can comprise administering to a subject in need thereof, a therapeutically effective amount of the composition disclosed herein or the pharmaceutical composition disclosed herein. For example, disclosed herein are methods of inducing apoptosis of a cancer cell, the methods can comprise administering to a subject a therapeutically effective amount of the composition comprising: acetaminophen or an analog thereof; and CYP2E1 inhibitor or the pharmaceutical composition comprising: acetaminophen or an analog thereof; and CYP2E1 inhibitor. Further disclosed herein are methods of inducing apoptosis of a cancer cell, the methods can comprise administering to the subject a therapeutically effective amount of acetaminophen or an analog thereof and a therapeutically effective amount of a CYP2E1 inhibitor. In some aspects, the cancer cell or cancer cells can be mammalian cells. In some aspects, the methods can include contacting a cell or tissue or administering to a subject in need thereof, a therapeutically effective amount of the composition disclosed herein or the pharmaceutical composition disclosed herein. In some aspects, the therapeutically effective amount of acetaminophen can be administered to the subject, concurrently, simultaneously, or sequentially with the therapeutically effective amount of a CYP2E1 inhibitor.

Disclosed herein are methods of treating cancer in a subject. In some aspects, the methods can comprise administering to the subject a therapeutically effective amount of the composition disclosed herein or the pharmaceutical composition disclosed herein. For example, disclosed herein are methods of treating cancer in a subject, the methods can comprise administering to the subject a therapeutically effective amount of acetaminophen or an analog thereof and a therapeutically effective amount of a CYP2E1 inhibitor. Further, disclosed herein are methods of treating cancer in a subject, the methods can comprise administering to the subject a therapeutically effective amount of the composition comprising: acetaminophen or an analog thereof; and CYP2E1 inhibitor or the pharmaceutical composition comprising: acetaminophen or an analog thereof; and CYP2E1 inhibitor. In some aspects, the therapeutically effective amount of acetaminophen can be administered to the subject, concurrently, simultaneously, or sequentially with the therapeutically effective amount of a CYP2E1 inhibitor. The cancer can be any cancer. In some aspects, the cancer can be a primary or secondary tumor. In an aspect, the cancer can be a metastatic tumor. In other aspects, the primary or secondary tumor can be within the patient's breast, lung, lung or liver. In yet other aspects, the cancer has metastasized. In some aspects, the cancer may originate in the breast and metastasize to one or more of the following sites: the breast, lung, liver or bone.

Disclosed herein are methods of treating, preventing or reducing fever in a subject. In some aspects, the methods can comprise administering to the subject a therapeutically effective amount of the composition disclosed herein or the pharmaceutical composition disclosed herein.

Disclosed herein are methods of treating, preventing or reducing pain in a subject. In some aspects, the methods can comprise administering to the subject a therapeutically effective amount of the composition disclosed herein or the pharmaceutical composition disclosed herein.

In some aspects, the methods disclosed herein can further comprise administering one or more cancer therapeutic agents. In some aspects, the one or more cancer therapeutic agents can be anti-PD-1 antibodies, anti-PD-L1 antibodies, cisplatin, carboplatin, paclitaxel, doxorubicin, or gemcitabine.

In some aspects, the methods can reduce or prevent acetaminophen-induced liver toxicity.

In some aspects, the methods can reduce or prevent an increase in one or more markers of liver toxicity. In some aspects, the one or more markers of liver toxicity can be aspartate transaminase (AST), alanine transaminase (ALT), lactate dehydrogenase (LDH), alkaline phosphatase.

In some aspects, the methods can reduce or prevent an increase in alanine transaminase levels compared to the alanine transaminase levels before administration of the composition or pharmaceutical composition.

In some aspects, the subject has cancer. In some aspects, the cancer can be lung cancer, Breast cancer, pancreatic cancer, esophageal cancer, colon cancer, prostate cancer, or liver cancer. In some aspects, the subject has been diagnosed with cancer prior to the administering step.

In some aspects, the patient can be diagnosed with pain or fever prior to the administering step.

In some aspects, a subject “in need thereof” can be an individual who has been diagnosed with, previously treated for, and/or suspected of having the disease or condition to be treated. With respect to prevention, the individual in need thereof may also be an individual who is at risk for a disease or condition (e.g., a family history of the condition, life-style factors indicative of risk for the condition, etc.).

In some aspects, the disease or condition can be accompanied by a fever and/or pain. In some aspects, the disease or condition can be accompanied by inflammation. In some aspects, the acetaminophen or analog thereof and/or formulation comprising the acetaminophen or analog thereof can reduce the severity of one or more symptoms associated with a disease or condition that is responsive to acetaminophen or analog thereof by at least about any of 10%, 20%, 30%, 40%, 50%, 60%, 70%, 80%, 90%, 95%, or 100% compared to the corresponding symptom in the same subject prior to treatment or compared to the corresponding symptom in other subjects not receiving the acetaminophen or analog thereof and/or formulation.

In some aspects, the pain can be associated with pain in the skin or mucosa. In some aspects, the pain can be associated with injury, infection, thermal injury, radiation, cancer or other causes. In some aspects, pain can be associated with sunburns, inflammation, viral infection or mucosal injury. In an aspect, the methods disclosed herein can be used to treat or prevent pain associated with dermal tissue, subdermal tissue, muscosal membranes or any combination thereof.

Mucosa is a membrane that lines various cavities of the body and covers the surface of internal organs. It can be continuous with the skin. Examples of mucosa continuous with the skin at various body openings, include but are not limited to eyes, ears, inside the nose, inside the mouth, lip, the urethral opening and the anus. Additional examples of mucosa include but are not limited to bronchial mucosa and the lining of vocal folds; endometrium (mucosa of the uterus); esophageal mucosa; gastric mucosa; intestinal mucosa; nasal mucosa; olfactory mucosa; oral mucosa; penile mucosa; vaginal mucosa; frenulum of tongue; tongue; and anal canal.

Disclosed herein, are methods of treating a patient having pain. The pain can be from any cause including but not limited to acute and chronic pain. In some aspects, the pain can be due to burns, a thermal injury, inflammation, ischemic injury (e.g., myocardial or cerebral), neuronal injury. In some aspects, the compositions disclosed herein can be administered to the subject prophylactically to prevent or reduce post-operative pain. Examples of pain include but are not limited to post-surgical pain, post-operative pain (including dental pain), migraine, headache and trigeminal neuralgia, pain associated with burn, wound or kidney stone, pain associated with trauma (including traumatic head injury), neuropathic pain (e.g., peripheral neuropathy and post-herpetic neuralgia), pain associated with musculo-skeletal disorders, strains, sprains, contusions, fractures, such as myalgia, rheumatoid arthritis, osteoarthritis, cystitis, pancreatitis, inflammatory bowel disease, ankylosing spondylitis, sero-negative (non-rheumatoid) arthropathies, non-articular rheumatism and peri-articular disorders, and pain associated with cancer (including “break-through pain” and pain associated with terminal cancer). Examples of pain with an inflammatory component (in addition to some of those described above) include but are not limited to rheumatic pain, pain associated with mucositis, and dysmenorrhea. In some aspects, the methods and formulations of the present invention can be used to treat, reduce or prevent of post-surgical pain and/or cancer pain. In some aspects, the methods and compositions disclosed herein can be used to treat, reduce, or prevent pain that is associated with surgery, trauma, osteoarthritis, rheumatoid arthritis, lower back pain, fibromyalgia, postherpetic neuralgia, diabetic neuropathy, HIV-associated neuropathy and complex regional pain syndrome.

In some aspects, the administration of any of compositions described herein can reduce one or more of the symptoms of any of the diseases, disorders or conditions disclosed herein. In some aspects, the condition can be pain or fever. In some aspects, the administration of any of compositions described herein can reduce one or more of the symptoms of pain or fever. In some aspects, the one or more of the symptoms can be reduced for a period of at least 15 minutes to about 30 minutes. In some aspects, the one or more of the symptoms can be reduced for a period of at least 1 hour. In some aspects, the one or more of the symptoms can be reduced for a period of at least three hours.

The therapeutically effective amount or dosage of the acetaminophen or an analog thereof, and a CYP2E1 inhibitor used in the methods as disclosed herein applied to mammals (e.g., humans) can be determined by one of ordinary skill in the art with consideration of individual differences in age, weight, sex, other drugs administered and the judgment of the attending clinician. Variations in the needed dosage may be expected. Variations in dosage levels can be adjusted using standard empirical routes for optimization. The particular dosage of a pharmaceutical composition to be administered to the patient will depend on a variety of considerations (e.g., the severity of the cancer symptoms), the age and physical characteristics of the subject and other considerations known to those of ordinary skill in the art. Dosages can be established using clinical approaches known to one of ordinary skill in the art.

The duration of treatment with any composition provided herein can be any length of time from as short as one day to as long as the life span of the host (e.g., many years). For example, the compositions can be administered once a week (for, for example, 4 weeks to many months or years); once a month (for, for example, three to twelve months or for many years); or once a year for a period of 5 years, ten years, or longer. It is also noted that the frequency of treatment can be variable. For example, the present compositions can be administered once (or twice, three times, etc.) daily, weekly, monthly, or yearly.

Dosages of a CYP2E1 inhibitor can be in the range of 0.001 mg to 1 to 10,000 mg/day or 0.01 mg/mL to 1,000 mg/mL. In some aspects, the dosage of fomepizole can be 0.001 mg to 1 to 10,000 mg total or any amount in between or 0.01 mg/mL to 1,000 mg/mL. In some aspects, the dosage of propylene glycol can be 0.001 mg to 1 to 10,000 mg total or any amount in between or 0.01 mg/mL to 1,000 mg/mL. In some aspects, the dosage of disulfiram can be 0.001 mg to 1 to 10,000 mg total or any amount in between or 0.01 mg/mL to 1,000 mg/mL. In some aspects, the dosage of indazole can be 0.001 mg to 1 to 10,000 mg total or any amount in between or 0.01 mg/mL to 1000 mg/mL. In some aspects, the dosage of diallyl sulfide can be 0.001 mg to 1 to 10000 mg total or any amount in between or 0.01 mg/mL to 1,000 mg/mL. In some aspects, the dosage of clotrimazole can be 0.001 mg to 1 to 10,000 mg total or any amount in between or 0.01 mg/mL to 1,000 mg/mL. In some aspects, the dosage of isoniazide can 0.001 mg to 1 to 10,000 mg total or any amount in between or 0.01 mg/mL to 1,000 mg/mL.

In some aspects, the therapeutically effective dose of acetaminophen or an analog thereof can be less when combined with one or more of the CYP2E1 inhibitors disclosed herein. In some aspects, the administration of acetaminophen or an analog thereof, a CYP2E1 inhibitor, and one or more anti-cancer therapeutic agents can be synergistic.

The total effective amount of the compositions as disclosed herein can be administered to a subject as a single dose, either as a bolus or by infusion over a relatively short period of time, or can be administered using a fractionated treatment protocol in which multiple doses are administered over a more prolonged period of time. Alternatively, continuous intravenous infusions sufficient to maintain therapeutically effective concentrations in the blood are also within the scope of the present disclosure.

The compositions described herein can be administered in conjunction with other therapeutic modalities to a subject in need of therapy. The present compounds can be given to prior to, simultaneously with or after treatment with other agents or regimes. For example, acetaminophen or an analog thereof, a CYP2E1 inhibitor disclosed herein can be administered in conjunction with standard therapies used to treat cancer (or pain or fever). In some aspects, any of the compositions or pharmaceutical compositions described herein can be administered or used together with chemotherapy.

In some aspects, acetaminophen or an analog thereof and the CYP2E1 inhibitor can be co-formulated.

Any of the compounds or compositions described herein can be administered as a term “combination.” It is to be understood that, for example, acetaminophen or an analog thereof can be provided to the subject in need, either prior to administration of a CYP2E1 inhibitor, concomitant with administration of the CYP2E1 inhibitor, or any combination thereof (co-administration) or shortly thereafter.

In some aspects of the methods disclosed herein, a therapeutically effective amount of acetaminophen or an analog thereof and a CYP2E1 inhibitor can be administered concurrently, simultaneously, or sequentially. In some aspects, fomepizole has a longer half-life than acetaminophen. As a result, fomepizole may be given on a various schedules (most commonly twice daily) in order to maintain therapeutic serum concentrations. During the time when fomepizole has therapeutic serum concentrations, acetaminophen may be given on an independent schedule, such as every 4-6 hours.

### Articles of Manufacture

The compositions and pharmaceutical compositions described herein can be packaged in a suitable container labeled, for example, for use as a therapy to treat cancer (or fever or pain) or any of the methods disclosed herein. Accordingly, packaged products (e.g., sterile containers containing the composition described herein and packaged for storage, shipment, or sale at concentrated or ready-to-use concentrations) and kits, including at least acetaminophen or an analog thereof; and a CYP2E1 inhibitor as described herein and instructions for use, are also within the scope of the disclosure. A product can include a container (e.g., a vial, jar, bottle, bag, or the like) containing the composition described herein. In addition, an article of manufacture further may include, for example, packaging materials, instructions for use, syringes, buffers or other control reagents for treating or monitoring the condition for which prophylaxis or treatment is required. The product may also include a legend (e.g., a printed label or insert or other medium describing the product's use (e.g., an audio- or videotape)). The legend can be associated with the container (e.g., affixed to the container) and can describe the manner in which the compound therein should be administered (e.g., the frequency and route of administration), indications therefor, and other uses. The compositions can be ready for administration (e.g., present in dose-appropriate units), and may include a pharmaceutically acceptable adjuvant, carrier or other diluent. Alternatively, the compounds can be provided in a concentrated form with a diluent and instructions for dilution.

In some aspects, acetaminophen or an analog thereof; and the CYP2E1 inhibitor can be co-packaged.

### EXAMPLES

### Example 1: Fomepizole Combined with NAC is Effective at Preventing AAP Toxicity

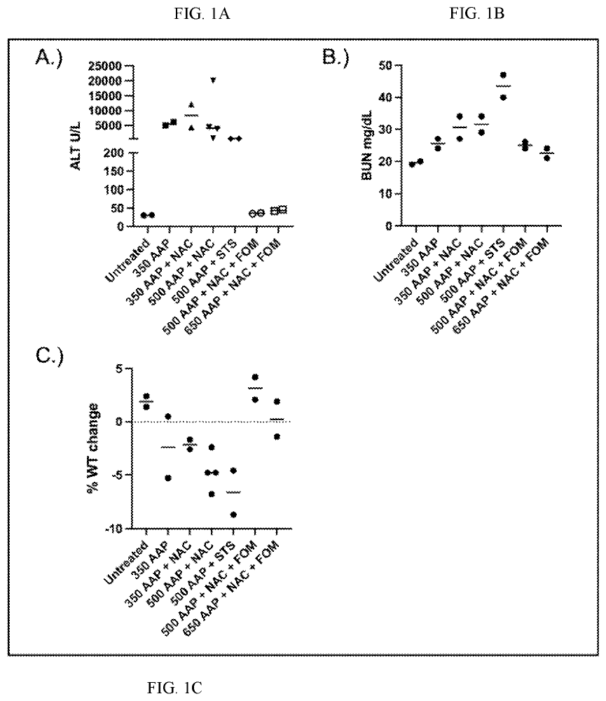

Rescue regimens for preventing toxicity of high dose AAP were evaluated. The results demonstrated that fomepizole+NAC prevented any detectable evidence of toxicity using AAP doses as high at 650 mg/kg as measured by ALT, BUN, and weight changes 24 hours after treatment (FIG. 1). The lack of efficacy of concurrent NAC to prevent AAP-induced hepatotoxicity is an unexpected result. Mechanistically, NAC neutralizes free radicals that have been generated from AAP toxicity. It is thus possible that NAC is more effective when given at delayed time points after AAP treatment, e.g., when free radicals have already been generated. On the other hand, fomepizole, a cyp2e1 inhibitor, prevents the formation of free radicals (e.g., NAPQI) and thus may be most effective when administered concurrently with AAP.

### Example 2: Fomepizole and NAC do not Prevent AAP Cytotoxicity Towards the Tumor In Vitro

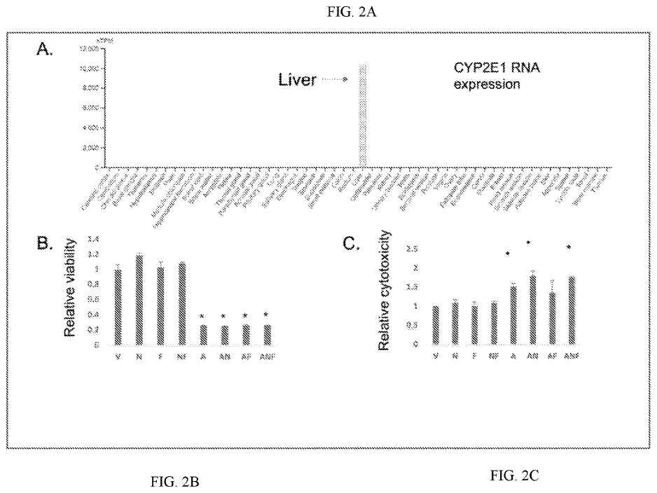

Data from the Protein Atlas demonstrates that CYP2E1 is expressed exclusively in the liver (FIG. 2A). Next, it was evaluated whether fomepizole, a CYP2E1 inhibitor, selectively prevents AAP cytotoxicity in the liver but not tumor cells in vitro. It has previously been published that fomepizole reverses AAP toxicity against hepatocytes (Akakpo J Y, et al. Hum Exp Toxicol. 2018). The effects of fomepizole on AAP cytotoxicity against EF43.fgf4 triple negative breast cancer cells in vitro was determined. Using the MTT viability assay, the results show that fomepizole and NAC do not reverse AAP cytotoxicity in tumor cells (FIG. 2B). Additionally, an LDH release assay similarly demonstrated that AAP-induced tumor cell death was not reversed by concurrent treatment with NAC and/or fomepizole (FIG. 2C).

### Example 3: Fomepizole does not Reverse AAP Anti-Tumor Efficacy In Vivo

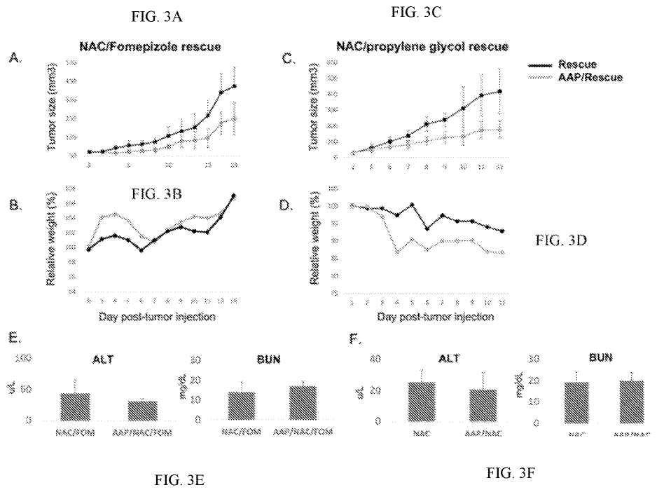

The anti-tumor efficacy of AAP was tested using a NAC (100 mg/kg)/propylene glycol (10%) rescue regimen. NAC could not be used alone rescue due to liver toxicity (FIG. 1). Propylene glycol, in addition to functioning as a diluent that helps solubilize AAP, is a CYP2E1 inhibitor that prevents AAP toxicity (Thomsen, M. S., et al. Pharmacol Toxicol. 1995 June; 76 (6): 395-9). The results show that AAP (500 mg/kg) decreased tumor growth in a 4T1 orthotopic breast cancer tumor model. While there was no liver toxicity, the mice did lose about 10% of body weight from the treatment regimen (FIGS. 3C, 3D).

Based on the toxicity studies (FIG. 1), AAP at doses of up to 650 mg/kg could be given with NAC and fomepizole rescue with no weight loss or liver toxicity. Thus, the experiments with 4T1 breast tumors were repeated using fomepizole and NAC concurrently to rescue at a higher dose of AAP (650 mg/kg) (FIGS. 3A, 3B, and 3E). AAP anti-tumor efficacy was preserved when administered with fomepizole/NAC rescue. Further, the mice did not have any weight loss (FIG. 3B).

Upon animal euthanasia at the conclusion of the experiment, serum ALT and BUN levels were measured in both treatment groups (rescue alone and rescue with AAP). Levels of ALT and BUN were normal, thus demonstrating that AAP administered along with fomepizole or NAC and propylene glycol did not induce any liver or kidney toxicity of AAP (FIGS. 3E, 3F).

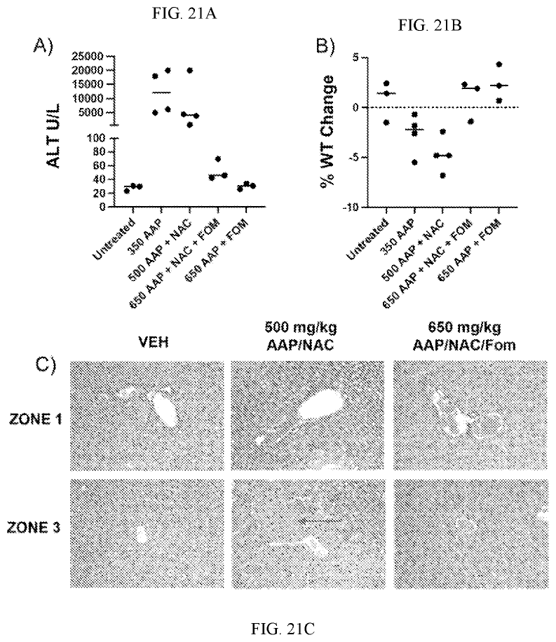

Additionally, FIG. 21 shows that NAC is inadequate at preventing AAP toxicity but fomepizole can prevent AAP toxicity. FIGS. 21A-C show BALB/c mice were treated with indicated dose of AAP+/−NAC (100 mg/kg) and/or fomepizole (30 mg/kg) all concurrently. 24 h later serum and liver histology were analyzed.

### Example 4: High Dose Acetaminophen with N-Acetylcysteine Rescue Inhibits M2 Polarization of Tumor Associated Macrophages

High dose acetaminophen (AAP) with n-acetylcysteine (NAC) rescue is among the few treatments that has shown a signal of activity in phase I trials (21% response rate, 3/14 assessable patients) without achieving dose limiting toxicity that has not progressed to evaluation in later line studies. It has been shown that high dose AAP selectively depletes glutathione in the liver but not the tumor, suggesting that NAC, the FDA-approved antidote for AAP toxicity and a glutathione precursor, may selectively rescue the normal liver but not the tumor from high dose AAP. While the anti-tumor effects of AAP in combination with NAC appear to not be mediated by glutathione depletion and free radical injury, the mechanism of anti-tumor effects of AAP/NAC has not been definitively characterized.

Methods. In vitro the effects of vehicle, AAP, or AAP in combination with NAC were evaluated on bone marrow-derived macrophages polarized to M2 and M1 phenotypes using IL-4 or IFN/LPS, respectively. Effects of AAP on IL-4/STAT6 (M2) or IFN/LPS/STAT1 (M1) signaling, and downstream gene and protein expression were studied. In vivo, effects of AAP in combination with NAC on tumor size and tumor associated macrophage polarization were characterized using cytokine ELISA of tumor lysate and flow cytometry.

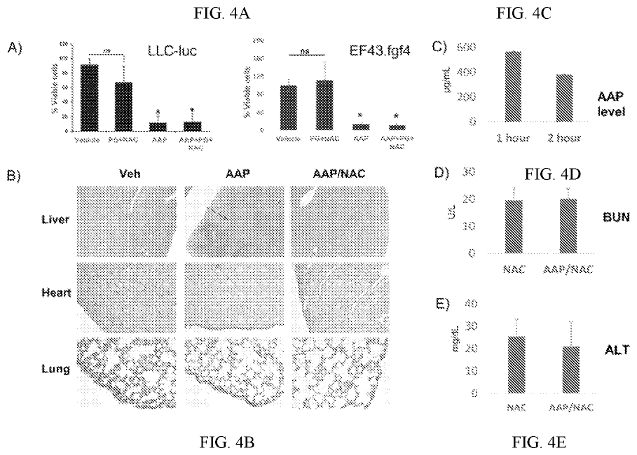

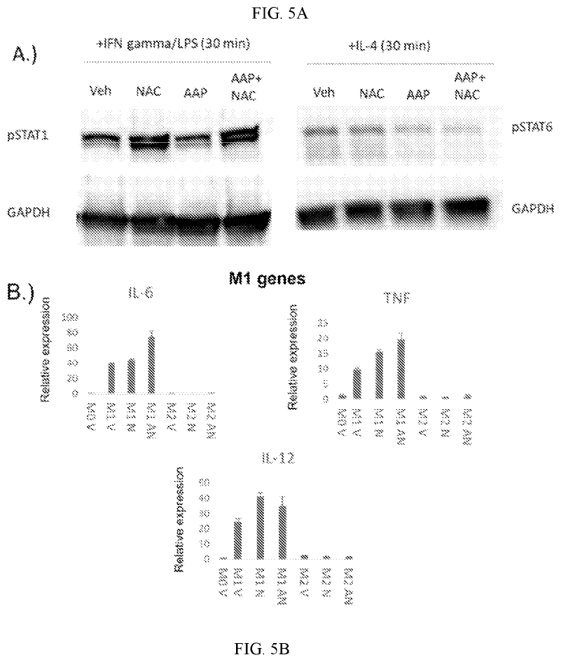

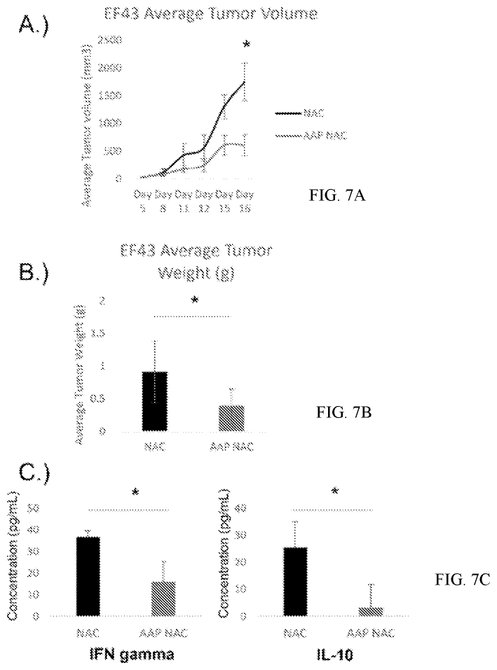

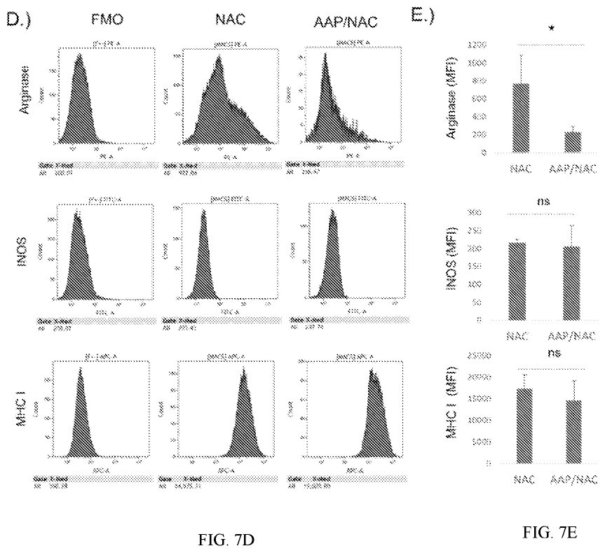

Results. NAC reversed AAP-induced toxicity in the normal liver but did not reverse AAP cytotoxicity against tumor cells in vitro. Administration of AAP in combination with NAC selectively inhibited IL-4-induced STAT6 phosphorylation but not IFN/LPS-induced STAT1 phosphorylation. Downstream, the administration of AAP in combination with NAC inhibited IL-4 induction of M2-associated genes (e.g., CCL24, YM1 and arginase) and proteins (e.g., arginase, PD-L2, and CD206). However, the administration of AAP in combination with NAC did not inhibit IFN/LPS induction of M1-associated genes (e.g., IL-6, TNF, IL-12) and proteins (e.g., INOS, PD-L1, MHC I, and CD64). In vivo, the administration of AAP in combination with NAC inhibited tumor growth in EF43.fgf4 and 4T1 triple negative breast tumors. Flow cytometry of tumor-associated macrophages revealed that the administration of AAP in combination with NAC selectively inhibited M2 but not M1 polarization. ELISA of tumor lysate demonstrated relatively stable expression of IFN gamma but markedly suppressed expression of IL-10 in the tumor immune microenvironment. See, FIGS. 4 to 7.

to The results show that at high doses of AAP administered in combination with NAC has profound effects on the tumor immune microenvironment that can facilitate immune-mediated inhibition of tumor growth.

High dose AAP in combination with NAC has demonstrated efficacy and safety in early phase clinical trials. As described herein, the results demonstrate that AAP in combination with NAC alters the tumor immune microenvironment by inhibiting M2 polarization of tumor associated macrophages.

While at standard doses AAP is not thought to have anti-inflammatory properties, the results show that high dose AAP in combination with NAC has a profound effect on anti-tumor immunity. This effect appears to be mediated by selective inhibition of STAT6, which mediates M2 polarization, relative to STAT1, which mediates M1 polarization. Importantly, high dose AAP in combination with NAC was well tolerated and did not induce any detectable liver toxicity in the treated mice.

In conclusion, the results identified a free radical independent mechanism of anti-tumor activity of high dose AAP that is not reversed by NAC, the traditional antidote for AAP toxicity.

### Example 5: Generation of Spheroids from Both Established and Primary NSCLC Cells

Tumor spheroids grown in serum free CSC media are known to be enriched in cells with a CSC phenotype (Ishiguro T, et al. Cancer Sci. 2017; 108 (3): 283-9). H460 NSCLC spheroids express increased levels of the CSC marker CD133. 3D-tumor spheroids of three established human NSCLC cell lines (e.g., H460, A549, and H1975) with representative genetic abrasions observed in NSCLC patients (e.g., KRAS; KRAS; and EGFR, L858R, and T790M, respectively) were cultured. However, acetaminophen (AAP) established cell lines that have been in passage for years show genetic and morphological drift and hence fail to represent the original tumor from which they were derived (Torsvik A, et al. U-251 revisited: genetic drift and phenotypic consequences of long-term cultures of glioblastoma cells. Cancer Med. 2014; 3 (4): 812-24). To provide strengthened evidence of the validity of this approach, three primary human NSCLC-derived cells were obtained which are representative for common oncogenic driver mutations involving EGFR (CUTO 25, EGFR p.E746_A750del) and ALK (CUTO 29, EML4 (exon 6) ALK (exon 19) kinases. The genetic mutations shown were identified by sequencing.

CD133 expression in monolayer versus spheroids. H460 NSCLC cells were grown in monolayer or as spheroids and analyzed with immune-blotting.

### Example 6: Characterization of CSC-Phenotype in NSCLC Cells

CD133 is a widely used lung CSC marker (Zakaria N, et al. Front Oncol. 2017; 7:80). In order to validate the use of CD133 as a CSC marker in NSCLC an in vitro limiting dilution assay (LDA) was performed. H460 NSCLC cells were grown in CSC medium and flow sorted for CD133 high and low cells. Sorted cells were plated into 96-well plate at limiting dilutions (0→128 cells/well) and assessed for spheroid formation (50-150 μM) after a 4-day growth period. The results were analyzed according to the ELDA protocol (Hu Y and Smyth G K. J Immunol Methods. 2009; 347 (1-2): 70-8). A 9-fold increase in the spheroid forming frequency (SFF) in the CD133 high versus low cells was observed, supporting the use of CD133 as a CSC marker in NSCLC. Similar results were obtained using KRAS mutant A549 NSCLC cells (14-fold increase in SFF in CD133 high cells) and the patient derived EML4-ALK translocated CUTO 29 cell line (13-fold increase in SFF in CD133 high cells), demonstrating that CD133 functions as a CSC marker in NSCLC with diverse driver mutations.

Next, in vivo LDA was used to evaluate whether CD133 can be used as a marker of tumor initiating cells in NSCLC. H460 cells were flow-sorted to separate CD133 high and CD133 low cells. The cells were implanted in NCr nude mice in a limiting dilution, implanting 2K, 5K, 10K, 50K and 100K cells subcutaneously (4 mice per condition). Tumor size over 150 mm3 was assessed on day 55 (yes/no). Using ELDA software, it was determined that CD133 high cells had a 5-fold increase in tumor initiating cell frequency compared to CD133 low cells.

In vitro and in vivo limiting dilution assays. H460 NSCLC cells were grown in CSC media and flow sorted for CD-133 high and CD-133 low cells. The CD133 high and CD133 low cells were plated at limiting dilutions (0→128 cells/well) and assessed for spheroid formation (yes/no). In bottom panel, the CD133 high and CD133 low cells were implanted into mice in limiting dilutions and assessed for tumor formation (yes/no). Data analyzed with ELDA software.

### Example 7: AAP Inhibits CSC Phenotype In Vitro

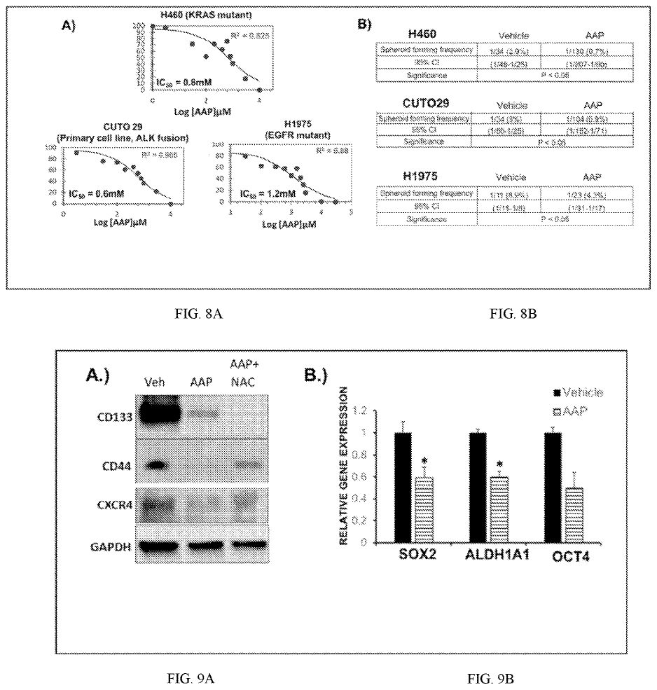

The results demonstrate that high dose AAP inhibits CSC phenotype in vitro, as evidenced by reduction in 3D tumor sphere formation (FIG. 8A), self-renewal (using a LDA) (FIG. 8B) and changes in CSC specific marker expression (FIG. 9).

The ability to form 3D tumor spheroids is a signature of CSC phenotype (Ishiguro T, et al. Cancer Sci. 2017; 108 (3): 283-9). NSCLC cells were plated into CSC medium along with increasing concentrations of AAP. Four days later the number of spheroids 50-150 μM were assessed. The number of spheroids in the AAP treated wells were normalized to vehicle and plotted using SigmaPlot (FIG. 8). The IC₅₀ value of H460 (established line with KRAS mutation) cells was 0.8 mM, for CUTO 29 (patient-derived EML4-ALK fusion) 0.6 mM and for H1975 (established line with EGFR mutation) 1.2 mM. These results show that AAP inhibits spheroid growth in NSCLC cells with diverse driver mutations.

Self-renewal: an in vitro limiting dilution assay (LDA) was used to assess self-renewal in AAP-treated NSCLC cells. H460, CUTO 29 and H1975 human NSCLC cells were grown as spheroids and treated with vehicle or 1 mM AAP for 24 hours. The cells were then plated in CSC medium for 4 days without any further AAP treatment and the wells with a spheroid 50-150 μM were counted (yes/no). The results demonstrated a 4-fold, 3-fold and 2-fold decrease in the spheroid forming frequency in the AAP-treated H460 CUTO 29, and H1975 cells, respectively (FIG. 8), demonstrating that AAP inhibits self-renewal in NSCLC cells.

Changes in the expression of CSC related proteins and genes upon treatment with AAP was evaluated. H460 NSCLC cells were cultured in spheroid medium, and treated with vehicle, AAP (1 mM) or AAP+NAC (1 mg/mL) for 24 hours. The cells were collected and subjected to immunoblotting. AAP markedly reduced expression of CD133, CD44 and CXCR4 in H460 cells, and this effect was not reversed by concurrent NAC (FIG. 9). Quantitative PCR was performed on CSCs treated with vehicle or AAP for 24 hours. Results demonstrated significant reduction in expression of the CSC associated genes SOX2 and ALDH1A1, as well as a non-significant trend towards decreased expression of OCT4.

In summary, the data provide evidence that AAP inhibits CSCs in vitro, an effect not reversed by NAC.

### Example 8: High Dose AAP Inhibits Tumor Growth and Spheroid Formation in Xenograft Models

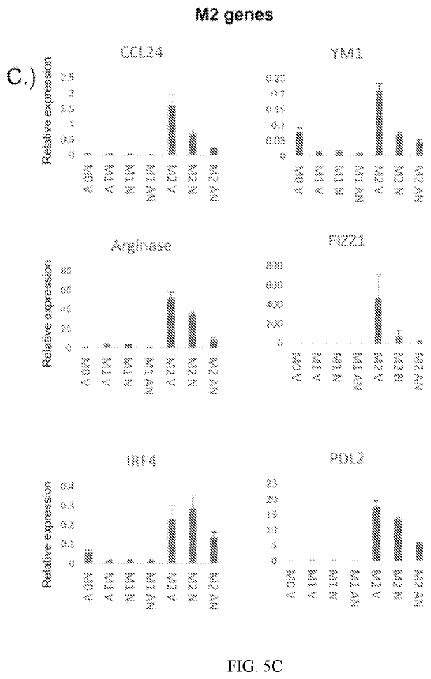

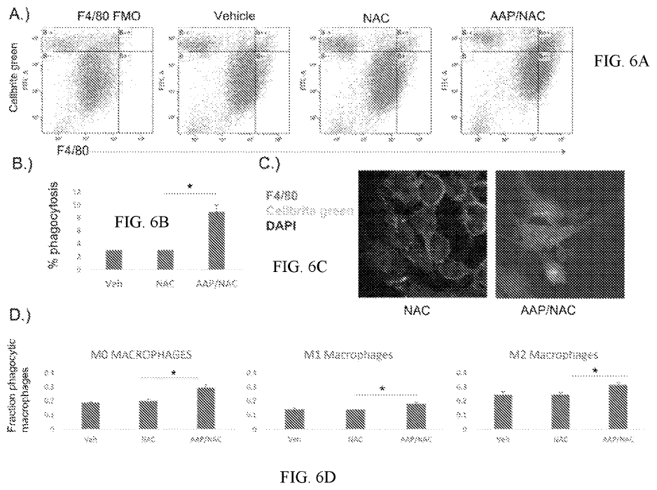

The data to demonstrate that high dose AAP impairs tumor growth (FIG. 10) and spheroid formation in cells isolated from tumor bearing xenografts (FIG. 6). These effects are not reversed by NAC.

As a functional test to evaluate the anti-CSC effects of AAP in vivo, H460 NSCLC cells were grown in CSC media and then flow-sorted to isolate CD133+ cells. The CSCs (10⁵ CD133+ cells) were implanted into nude mice, and upon formation of palpable tumors, mice were treated with vehicle, NAC (100 mg/kg) or AAP (350 mg/kg)+NAC (FIG. 10A). Results demonstrated that AAP+NAC impaired tumor growth in vivo. Further, there was no evidence of liver injury in the AAP+NAC treated mice, as demonstrated by ALT levels (FIG. 10B).

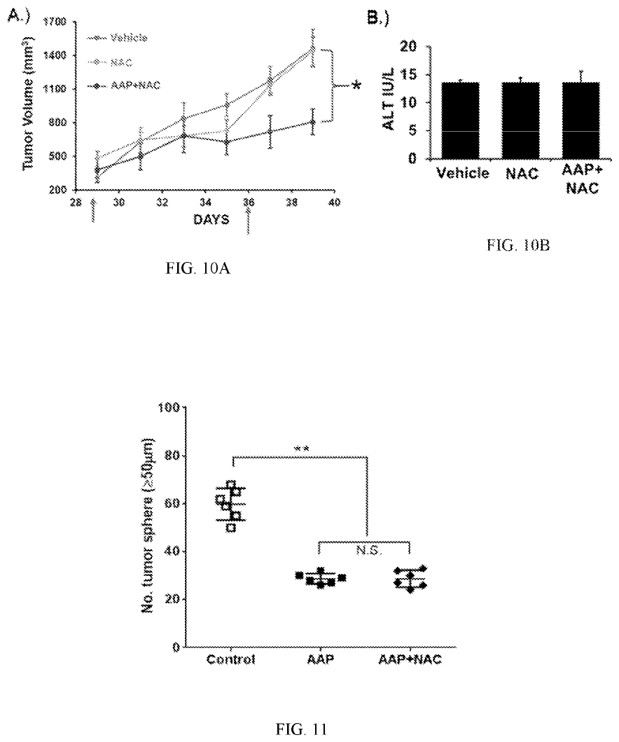

The ability of AAP to inhibit spheroid growth in a rat subcutaneous xenograft model of human H460 NSCLC cells was assessed. 2.5×10⁷ H460 cells were injected subcutaneously into athymic rats, and tumors were allowed to form. Tumor-bearing rats were treated with vehicle, AAP (600 mg/kg PO via oral gavage), or AAP+NAC (1000 mg/kg IV). Four days after drug treatment, tumors were digested in collagenase at 37° C. for 30 min and filtered through 60 μm strainer to obtain single cell suspension for in vitro tumorsphere formation assay. When grown in stem cell media, tumor cells from the AAP-treated or AAP+NAC-treated rats had significantly impaired ability to form 3D tumor spheroids relative to the tumors from the untreated rats supporting the anti-CSC properties of AAP in vivo, demonstrating that NAC does not interfere with the anti-CSC effects of AAP (FIG. 11).

Overall, AAP depletes CSCs in xenografts and inhibits CSCs in vivo, an effect not reversed by treatment with NAC.

### Example 9: AAP is a STAT3 Inhibitor

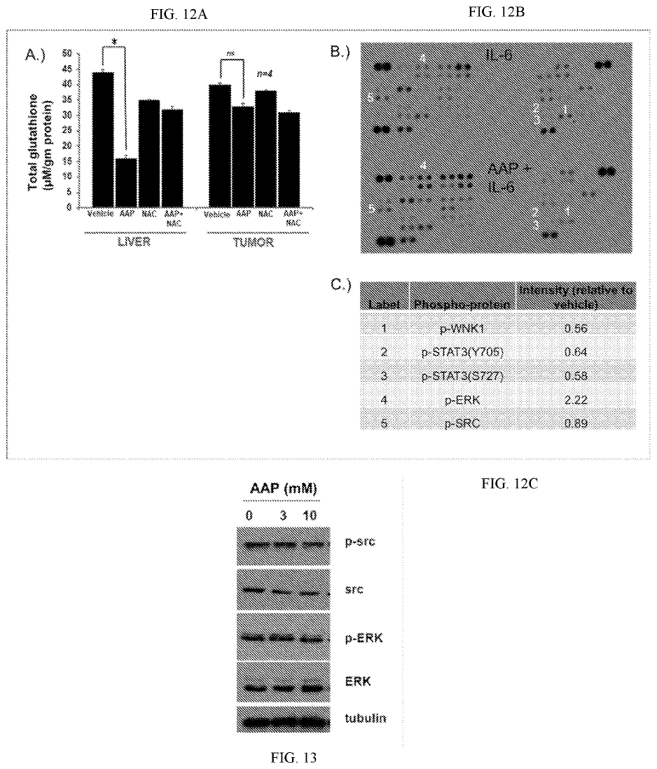

The results described herein demonstrate that high dose AAP inhibits phosphorylation of STAT3 at Y705 and S727 (FIGS. 12, and 14) as well as downstream targets of STAT3 (FIG. 10).

The presumed mechanism of anti-tumor activity of AAP is analogous to the mechanism of AAP-induced liver toxicity, which is glutathione depletion and resulting free radical injury (Kobrinsky N L, et al. Cancer Invest. 1996; 14 (3): 202-10). To test this hypothesis, H460 tumor bearing mice were treated with AAP (500 mg/kg) and/or NAC (100 mg/kg). Four hours later the mice were sacrificed and the tumors and livers analyzed for glutathione content. Interestingly, glutathione was selectively depleted in the liver but not the tumor (FIG. 12A). This result is consistent with findings in ovarian cancer and suggests a free-radical independent mechanism of AAP's anti-tumor activity (Wu Y J, et al. Anticancer Res. 2013; 33 (6): 2391-400).

To better understand the mechanisms of AAP's anti-CSC activity, an unbiased phospho-kinase array was performed to evaluate the expression of 41 phospho-proteins in response to AAP treatment. H460 NSCLC cells were treated for 2 hours with 10 mM vehicle or AAP. The media was changed and replaced with fresh media containing 20 ng/ml IL-6. Twenty minutes later the cells were collected and phospho-kinase array was performed according to the manufacturer protocol (R&D). Signal was quantified using UN-SCAN-IT software. The analysis focused on phospho-proteins associated with CSC growth/self-renewal. p-ERK levels were increased 2.2-fold in response to AAP treatment, while p-SRC levels were relatively unchanged (FIGS. 12B, C). However, no changes in p-SRC and p-ERK were observed with immune-blotting (FIG. 13). Additionally, WNK1 levels were suppressed in response to AAP treatment (relative intensity 0.56 of AAP-treated cells compared to vehicle). While there is some evidence that WNK1 is a target for glioblastoma stem like cells (Chen W, et al. Oncotarget. 2018; 9 (43): 27197-219), a strong link between WNK1 and lung CSCs has not been identified. The phospho-kinase array also demonstrated that AAP inhibited phosphorylation of STAT3 at both Y705 and S727 (relative intensity 0.64 and 0.58 compared to vehicle, respectively). STAT3 is a transcription factor that has been relatively well validated as a regulator of lung CSC-related gene expression and phenotype (Shao C, et al. Clin Cancer Res. 2014; 20 (15): 4154-66; and Li Y, et al. Proc Natl Acad Sci USA. 2015; 112 (6): 1839-44).

To validate the results of the phospho-kinase array, immune-blotting was performed. H460 cells were grown as spheroids in CSC medium treated with 1 mM AAP for indicated duration of time. The lysates were collected and analyzed by immune-blotting (FIG. 14A). AAP resulted in partial inhibition of phosphorylation of tyrosine 705 (Y705) within 2 hours. Near complete inhibition of phosphorylation was observed at 4 hours that persisted until 24 hours later (FIG. 14A).

Next, it was evaluated whether AAP-induced STAT3 inhibition is affected by concurrent NAC administration. H460 NSCLC cells were grown in spheroid medium and treated with Vehicle, AAP (1 mM) or AAP+NAC (1 mg/mL) for 24 hours. The cells were then collected and subjected to immune-blotting analysis. It was demonstrated that AAP inhibited pSTAT3 at both Y705 and S727. The anti-oxidant thiol NAC did not reverse AAP-induced STAT3 inhibition (FIG. 14B), demonstrating that ROS-independent mechanisms regulate AAP-induced STAT3 inhibition. AAP similarly inhibited STAT3 phosphorylation at Y705 and S727 in CUTO 29 cells, a patient-derived cell line with EML4-ALK (E6: A19) (FIG. 14C). These results show that AAP can inhibit STAT3 phosphorylation in NSCLC tumors with diverse driver mutations.

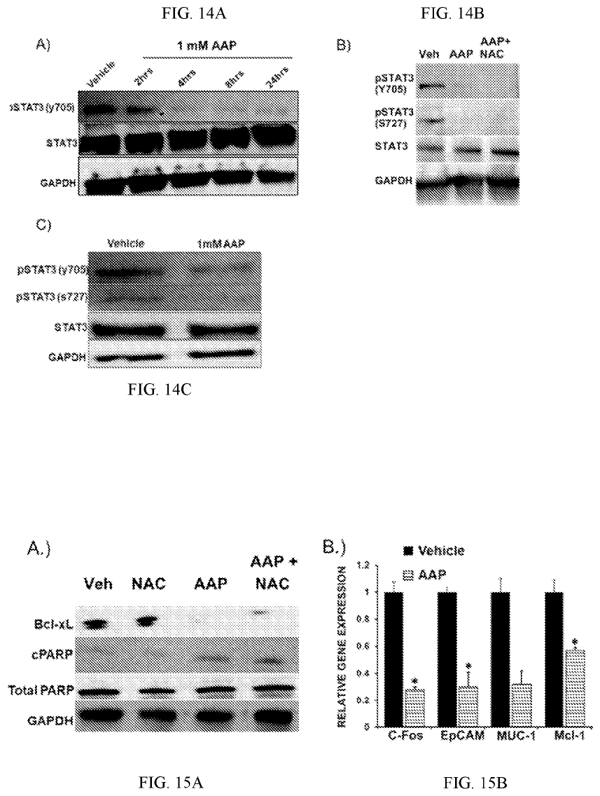

To evaluate for functional inhibition of STAT3 by AAP, genes and proteins downstream of STAT3 were analyzed. Spheroids grown in CSC medium were treated with AAP (1 mM) and/or NAC (1 mg/mL) for 24 hours. Bcl-xL expression was strongly inhibited by AAP. Given that Bcl-xL functions as an anti-apoptotic protein, the effect of AAP on cleaved PARP was further evaluated. Cleaved PARP had increased expression in AAP treated cells showing that the anti-CSC activity of AAP may proceed via the induction of apoptosis. Importantly, the pro-apoptotic effect of AAP was not reversed by NAC (FIG. 15). Using qPCR, it was demonstrated that MCL-1, C-FOS and EpCAM, genes whose expression is regulated by STAT3 (Yu H, et al. Nat Rev Cancer. 2014; 14 (11): 736-46), had decreased gene expression when cells were treated with AAP (FIG. 15).

In summary, AAP inhibits STAT3 at Y705 and S727, effects that are not reversed by concurrent treatment with NAC. AAP further inhibits expression of proteins downstream of STAT3.

### Example 10: AAP Inhibits CSC Marker Expression Via a STAT3 Dependent Mechanism

The results described herein demonstrate that the anti-CSC effects of AAP are lost in STAT3 knockdown cells (FIG. 16) showing that STAT3 may mediate the anti-CSC effects of AAP.

Next it was determined whether the anti-CSC activity of AAP occurs via modulation of STAT3 activity. Transient knockdown (KD) of STAT3 in H460 NSCLC cells was achieved using shRNA. AAP (1 mM) inhibited spheroid formation by >50% in scrambled H460 cells compared to vehicle control. However, AAP had no effect on spheroid formation in the STAT3 knockdown cells compared to vehicle control, demonstrating that AAP mediates its anti-CSC effects, in part, via STAT3 inhibition (FIG. 16A). At a molecular level, in further support of the spheroid formation findings, STAT3 KD cells showed inhibition of CSC marker CD133, phenocopying the effect of AAP. Also, AAP treatment alone inhibited CD133 expression in scrambled but not STAT3 KD cells compared to vehicle control (FIG. 16B). Hence, STAT3 is a direct target of AAP and plays an important role in mediating its anti-CSC properties.

### Example 11: The Role of STAT3 Inhibition in AAP's Anti-CSC Activity

Determine the molecular mechanisms of AAP-induced STAT3 inhibition in NSCLC. Y705 and S727 on STAT3 are regulated by distinct upstream signaling pathways (Huynh J, et al. Nat Rev Cancer. 2019; 19 (2): 82-96). The fact that AAP inhibits both Y705 and S727 (FIG. 14) raises the possibility that AAP directly binds to STAT3. Of note, S727 and Y705 are both located within the STAT3 transactivation domain; it will be tested whether AAP inhibits both phosphorylation sites via direct binding.

Results. Computational studies predict favorable and selective binding of AAP to STAT3.

Computational methods were used to predict interaction of ligands with proteins, and in particular binding selectivity, potency and site of binding on STAT3 was assessed. Sites of AAP binding on STAT3 were identified using an unbiased search algorithm, followed by a genetic algorithm-based docking and scoring study (Sankarayanarayanan N V, et al. Angew Chem Int Ed Engl. 2017; 56 (9): 2312-7) to derive favorable binding geometries, if any. The genetic algorithm-based study predicted a highly preferred binding pocket for AAP on STAT3 ( ), which allows formation of strong hydrogen bonding interactions with K626 and hydrophobic interactions with E625, D627, 1659, M660, D661, A662 and F716 ( ). These data showed that AAP was likely to bind STAT3 near the transactivation domain with high selectivity.

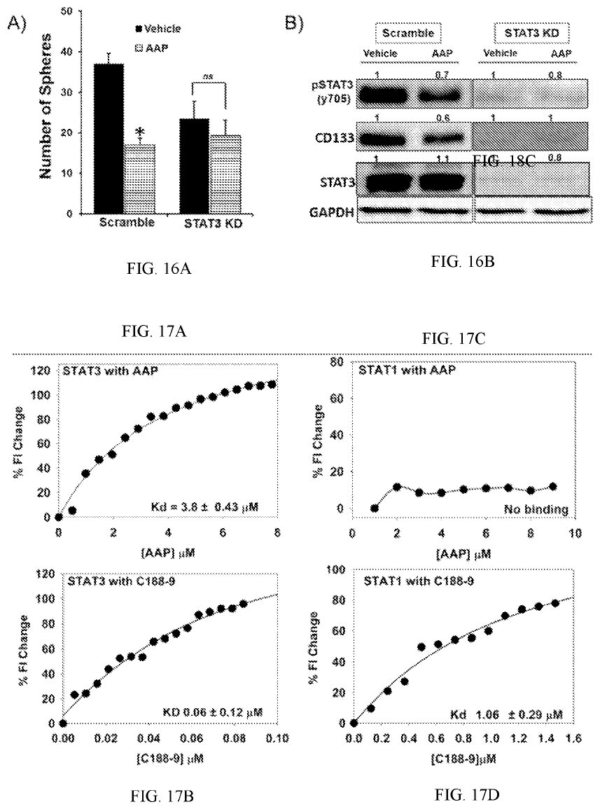

Biophysical techniques confirm computational predictions. To test computational predictions, spectrofluorimetry, which is a sensitive method that reports on changes in intrinsic protein fluorescence if a ligand binds with reasonable affinity (Parikh H H, et al. Pharm Res. 2000; 17 (5): 632-7) was used. The interaction can be used to measure the affinity of the complex. STAT3 (22 nM) was titrated with AAP at 25° C. while monitoring emission at 340 nm (MEX=280 nm) using PTI spectrofluorimeter (FIG. 17, top left panel). A robust saturable fluorescence signal was observed indicating the formation of STAT3-AAP complex. Non-linear regression using the quadratic binding equation yielded an affinity of 3.8 μM. Similar experiments with STAT1 showed no change in fluorescence (FIG. 17, top right panel). In contrast, the STAT3 inhibitor C188-9 (also known as TTI-101) that is being evaluated in clinical trials (NCT03195699), demonstrated direct binding to both STAT3 and STAT1, consistent with results of prior pre-clinical studies (Jung K H, et al. Clin Cancer Res. 2017; 23 (18): 5537-46) (FIG. 17, bottom panels). ibuprofen was used as a negative control, which showed no binding to STAT3 or STAT1 at concentrations up to 100 μM further providing evidence of the specificity of AAP scaffold's interaction with STAT3.

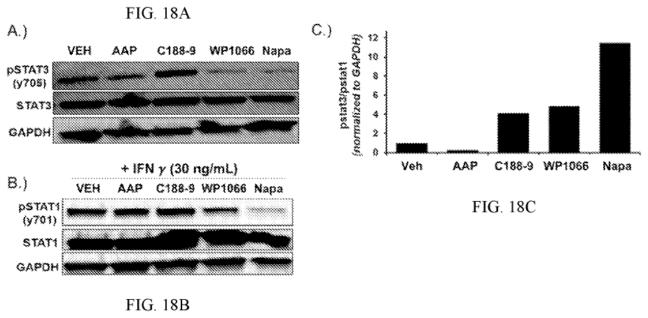

Next, it was evaluated whether AAP had a high degree of specificity for STAT3 over STAT1 using in situ studies and the results were compared with other STAT3 inhibitors being evaluated in clinical trials. H460 NSCLC cells were grown in CSC media and treated with AAP, C188-9, WP1066 or napabucasin for 4 hours prior to immune-blotting analysis. Results demonstrated that AAP, unlike other STAT3 inhibitors in clinical development, has a high degree of specificity for inhibition of STAT3 phosphorylation relative to STAT1 (FIG. 18), a finding consistent with our in vitro binding studies (FIG. 17). Specificity for STAT3 over STAT1 is desirable because STAT3 appears to be pro-tumorigenic and STAT1 plays an important role in the anti-tumor immune response (Avalle L, et al. JAKSTAT. 2012; 1 (2): 65-72).

STAT3 K626A constructs retain STAT3 WT function and retains CSC phenotype: Given the in silico findings that AAP binds STAT3 via a strong hydrogen bond at K626, a mutant, STAT3 K626A, was created and predicted to have suppressed affinity to AAP. CRISPR/cas9 technology was used to knock out STAT3 in A549 NSCLC cells. Subsequently, site directed mutagenesis was used to substitute a gene encoding alanine (DNA sequence GCA) for the wild type lysine (DNA sequence AAA) in a pcDNA vector. Sequencing confirmed successful creation of a STAT3 K626A mutant plasmid.

To determine whether STAT3 K626A promotes a CSC phenotype, STAT3 KO A549 NSCLC cells were transiently transfected with K626A and WT STAT3. STAT3 phosphorylation was similar between WT and K626A mutants, evidencing similar STAT3 activation status between the cells. Morphologically, sphere-forming capacity was rescued in both the WT and K626A STAT3 transfected cells. CD133 expression was similar in WT and K626A cells. These results demonstrate that the K626A mutant retains STAT3 function and maintains baseline CSC phenotype.

Methods. STAT3 SH2 domain protein purification. To assess for AAP-binding to STAT3 modified constructs, the SH2 domain of STAT3 (which contains the AAP-binding site, as predicted by in silico analysis) will be purified. Protein isolation will be performed for STAT3 K626K (WT), and STAT3 K626A using published methodologies (Asai A and Takakuma K. Methods Mol Biol. 2017; 1555:163-72). More specifically, the SH2 domain of STAT3 will be amplified using PCR and the product run on an agarose gel. STAT3 cDNA will be cut from the gel and isolated. Restriction enzymes will be used to digest the STAT3 cDNA and the cDNA will be ligated into a pET28a (+) plasmid that contains a His-Tag. The plasmid product will be transformed into E. coli DH5α competent cells. After amplification in LB medium, the plasmid will be extracted using a mini-prep kit for sequencing to confirm effective transformation of the modified STAT3 protein. The modified STAT3-containing E. coli will be sonicated and run on a His-Trap HP column. The purified STAT3 protein will be eluted using an imidazole gradient (Asai A and Takakuma K. Methods Mol Biol. 2017; 1555:163-72).

Validation of AAP binding site to STAT3. AAP binding to the purified STAT3 protein products (WT and K626A) will be analyzed using in vitro (microscale thermopheresis and spectrofluorometry) and cellular (cellular thermal shift assay) methodologies (Pingali P W Y, et al. Neoplasia. 2021; 23 (3): 348-59). In brief, spectrofluorimetry will be performed as described here (FIG. 17) using purified STAT3 protein. Fluorescence spectra of STAT3 and STAT1 (22 nM) will be recorded in the wavelength range of 300-400 nm and used to calculate binding affinities based on change in fluorescence (ΔFmax) at saturation.

Microscale thermopheresis (MST) experiments will be performed on a Monolith NT system in label-free mode (NanoTemper Technologies GmbH, Munich, Germany) using purified STAT3 protein. Sufficient concentrations (~0.05-1.5 μM) of STAT3 and (~0.005 to 20 μM) of AAP will be used to study changes in thermophoresis as a function of the ligand.

Cellular shift assay will be performed on intact cells. NSCLC cells (containing STAT3 WT or K626A) grown in CSC media will be treated with indicated drug for 6 hours. Cells will be heated to various temperatures (40, 45, 50, 55, and 60 degrees Celsius) for 10 minutes followed by lysis with RIPA buffer and semi-quantitation of protein using Western blotting. Ligand binding is suggested by increased signal of STAT3 protein on immune-blotting that results from ligand-induced protein stabilization at elevated temperatures.

If the in-silico models are correct, AAP will bind with high affinity to STAT3 WT but with low (or no) affinity to STAT3 K626A.

Determine if AAP's anti-CSC effects are mediated by STAT3. Because the results described herein show that STAT3 KD significantly attenuated AAP's ability to inhibit primary spheroids (FIG. 16), demonstrating that STAT3-dependent anti-CSC effects of AAP. To validate this result, STAT3-dependence of the comprehensive CSC phenotype will be determined and the rescue of functional STAT3 inhibition will be used to evaluate STAT3's role in AAP's anti-CSC properties.

Generation of cells with altered target (STAT3) level, structure, and/or function to rescue AAP's anti-CSC properties. To determine whether the anti-CSC activity of AAP is mediated by direct binding resulting in inhibition of phosphorylation, NSCLC cell lines with STAT3 constructs will be generated (Table 1).

Full length (WT) STAT3 (WT STAT3) will be used as a control.

Y705F: To evaluate if AAP functions via inhibition of STAT3 phosphorylation, a Y705F mutant will be used. STAT3 Y705F functions as dominant negative preventing STAT3 phosphorylation, dimerization and nuclear translocation/transcription of the target genes. It is expected that Y705F will phenocopy the effect of AAP and have suppressed baseline CSC phenotype. If AAP functions via inhibition of phosphorylation at Y705, then AAP treatment would have a diminished effect on Y705F mutant cells.

STAT3C: STAT3C is a genetically engineered mutant of STAT3 that is constitutively dimerized independent of Y705 phosphorylation. STAT3C will be used as a dominant positive to evaluate for “rescue” from the effects of AAP (Bromberg J F, et al. Cell. 1999; 98 (3): 295-303). If AAP functions via inhibition of STAT3 phosphorylation at Y705, then it is expected that AAP treatment will have a diminished anti-CSC effect in STAT3C cells as a result of circumvention of target inhibition.

K626A: To determine if AAP inhibits CSC phenotype via direct binding to STAT3, K626A mutants will be used. It is expected that K626A, which does not form a hydrogen bond to AAP thus potentially inhibiting binding, will act as loss-of-function mutant for AAP's effects on STAT3 (and CSCs).

Synthesis of constructs: Y705F and STAT3C constructs (Addgene) will be used. K626A mutants and full length (WT) STAT3 A549 will be used. The altered constructs will be transfected into Phoenix packaging cells and virus containing medium will be added to STAT3 KO H460 and A549 human NSCLC cells. Following 7-10 days of infection, the cells will be flow sorted to isolate the GFP+vector containing cells.

TABLE 1
STAT3 altered cells to be evaluated.

| | Anticipated | Anticipated phenotype with |
|---|---|---|
| STAT3 | CSC phenotype | AAP treatment (relative to |
| construct | with Vehicle. | Vehicle) |
| Full-length | Baseline CSC phenotype | Suppression of CSC |
| (WT) STAT3 | | phenotype |
| STAT3 Y705F | Suppressed CSC | Diminished/Absent response |
| (phospho- | phenotype (Dominant | to AAP on CSC phenotype |
| resistant) | negative effects); | compared to Veh (due to DN |
| | Phenocopy of AAP | effect on target) |
| | effect. | |
| STAT3C | Enhanced CSC | Diminished/Absent response |
| (Constitutively | phenotype | to AAP on CSC phenotype |
| dimerized and | | compared to Veh |
| activated) | | (circumventing target |
| “Rescue” | | inhibition) |
| STAT3 K626A | Baseline CSC phenotype | Diminished/Absent response |
| | | to AAP on CSC phenotype |
| | | compared to Veh. (due to lack |
| | | of AAP binding to STAT3) |

Examine comprehensive CSC phenotype in NSCLC following treatment with AAP and/or NAC. CSCs are endowed with the ability to a) self-renew, b) invade and migrate, c) retain ability to differentiate, and d) resist killing by conventional chemotherapy (Visvader J E and Lindeman G J. Nat Rev Cancer. 2008; 8 (10): 755-68). The effects of AAP and NAC on 3D spheroids, using the primary and established cell lines described and constructs listed in Table 1, will be examined. The cells will be grown in CSC media and analyzed for growth, self-renewal, invasion, and differentiation as described below. Effects of AAP on sensitivity to FDA-approved chemotherapy and targeted therapy drugs will be examined.

Spheroid formation ability: Spheroids will be grown in 96-well plate in serum-free stem cell media. The effects of AAP (1 μM→10 mM) and NAC alone or in combination on ability of single cells seeded at 100-500 cells/well to form spheroids (50-150 micron) in 5-7 days will be examined. The results will be plotted as percent of vehicle (DMSO) control.

CSC self-renewal: Two methods to study self-renewal will be used. Dual tandem screening is a method to assess self-renewal of CSCs (Patel N J, et al. ACS Chem Biol. 2014; 9 (8): 1826-33). The spheroids (50-150 micron) will be treated with vehicle or AAP (1 μM-10 mM) for 24 hours. Following which the spheroids will be washed off the drugs and single cell suspension will be prepared to be plated in fresh the fresh CSC media without any additional treatment. The cells will be propagated in 2°-4° spheroids to evaluate for sustained inhibition of spheroid formation in the absence of drug treatment.

A limiting-dilution assay will also be used. For this, a single cell suspension prepared from the primary spheroids treated with vehicle or AAP for 24 hours (as above) will be plated in CSC media without any further treatment in limiting-dilution (1-128 cells/well) concentrations and spheroid formation 50-150 micron (yes/no) in each well will be noted at day 7 after plating. The spheroid formation frequency will be analyzed using ELDA software (Hu Y and Smyth G K. J Immunol Methods. 2009; 347 (1-2): 70-8)) to yield tumor initiating cell frequency and confidence intervals.

Comprehensive analyses of CSC/self-renewal markers. CSCs represent a slightly heterogeneous population. Thus, a single marker may not fully be reflective of lung CSCs. Hence, a complement of markers will be used to determine effects of various treatments on CSC population. CD44 is a well described CSC marker in NSCLC, and is associated with a poor prognosis and increased tumor proliferation (Hu B et al. Oncol Lett. 2018; 15 (4): 5627-33). CD133 and aldehyde dehydrogenase (ALDH) are other accepted CSC markers in NSCLC. CD133 was validated as a CSC marker using an in vitro and in vivo LDA in NSCLC cells. In addition, the levels of NANOG, OCT4, and SOX2 will be examined as regulators of self-renewal. The Aldefluor test will be used to analyze ALDH levels by flow cytometry (Shao C, et al. Clin Cancer Res. 2014; 20 (15): 4154-66).

Cell migration and/or invasion through Matrigel (Boyden chamber assay) will be examined by loading ~10⁵ spheroid cells pretreated with vehicle or AAP on the top chamber and assessing the number of cells in the bottom chamber (per 40×HPF, Giemsa stain) at 12, 24, and 48 h. Induction of differentiation, wherein the dissociated spheres treated with vehicle or AAP, will be grown in collagen coated monolayer condition in presence of 2.5% FBS and expression of gelsolin and mad (upregulation; both general makers of differentiation) and MUC1 and CC10 (downregulation; lung progenitor cell markers) (Chang T H and Szabo E. Cancer Res. 2000; 60 (4): 1129-38) will be measured by western-blot (WB) at different time points 5-14 days following AAP treatment.

The expected results are summarized in Table 1. Decreased efficacy of AAP (+/−NAC) on comprehensive CSC phenotype in K626A mutant cells will confirm that AAP inhibits CSC phenotype via selective binding to the STAT3 transactivation domain.

Transfecting STAT3 constructs into STAT3KO cells. In the above methods, STAT3 constructs (Y705F, STAT3C, K626A) will be transfected into STAT3KO NSCLC cells. This methodology avoids the presence of WT STAT3 in the transfected cells. The results described herein demonstrate that transfection of WT STAT3 or STAT3 K626A into STAT3KO NSCLC cells re-stores capacity to form spheroids in CSC media. CRISPR/Cas9 KO can also be used, and requires selection and expansion of individual clones. CSCs are a minor subset, and thus the probability of selecting a CSC in this selection/expansion process may be low. If subsequent studies reveal that transfection of the various STAT3 constructs (Y705F, STAT3C, K626A) into STAT3KO cells fails to induce the expected CSC phenotype, then transfecting the constructs into parental cells can be carried out. Using this methodology, a suppressed (although not eliminated, given the persistent presence of WT STAT3 in the transfected cells) effect of AAP on the mutant cells is expected.

An additional alternative approach includes introducing sh-resistant vectors into the STAT3 constructs followed by transfection into shSTAT3KD cells as a means to minimize WT STAT3 expression (Massengill M T, et al. Methods Mol Biol. 2019; 1937:235-58).

Alternative mechanisms underlying the anti-CSC activity of AAP. Other approaches can be used to evaluate the mechanism of AAP's anti-CSC activity.

RNA sequencing. RNA sequencing with pathway analysis can be used to evaluate the effects of AAP on CSCs. In brief, H460 and A549 spheroids grown in CSC media will be treated for 24 hours with vehicle or AAP (1 mM). RNA will be isolated using an RNeasy plus minikit (Qiagen). The samples will be sequenced according to Illumina's sequencing-by-synthesis protocol. About 30 million 150 bp paired-end reads per sample will be obtained allowing for five samples multiplexed per lane. The analysis of data on Illumina's BaseSpace Sequence Hub will generate FASTQ files containing the sequence reads. Sequencing adapters will be removed using Trimmomatic (Bolger A M, et al. Bioinformatics. 2014; 30 (15): 2114-20). Reads will be aligned using the latest assembly of the human genome (GRCh38/hg38) using the STAR 2.7 aligner (Dobin A, et al. Bioinformatics. 2013; 29 (1): 15-21. Gene expression levels will be generated from the aligned reads using annotation-guided approaches. For this, gene and exon read counts will be obtained for each sample based on the last hg38 version of Ensembl transcriptome (v. 87) using the featureCounts v. 1.2.6 software (Liao Y, et al. Bioinformatics. 2014; 30 (7): 923-30). The new ‘Tuxedo’ pipeline will additionally be used to obtain full and alternatively spliced transcript assemblies and their relative abundances. The biological interpretation of genes responding to treatments will be performed using “gold standard” bioinformatics tools, such as DAVID Bioinformatics Resources 6.8 (Dennis G, Jr., et al. Genome Biol. 2003; 4 (5): P3, GSEA (Gene Set Enrichment Analysis) (Subramanian A, et al. Proc Natl Acad Sci USA. 2005; 102 (43): 15545-50), and commercial software Ingenuity (Ingenuity Systems, Redwood City, CA).

Evaluating mutations involved in acquired resistance to AAP. A technique that has been described to identify the “true target” of oncologic drugs is by evaluating acquired mutations at the development of resistance (Lin A, et al. Sci Transl Med. 2019; 11 (509)). HCT-116 cells are used because they harbor an increased mutation rate as a result of a defect in mismatch repair. HCT-116 cells will be treated with lethal concentrations of AAP to select for resistant clones. Resistant clones will be isolated and subjected to whole exome sequencing. It is expected that the isolated clones will develop mutations in the genes encoding the “true target” of AAP. Results will be validated using genetic knockdown and rescue experiments. Analysis will be performed.

Evaluation of WNK1 as putative target of AAP. In the phospho-kinase array data, it was demonstrated that WNK1 phosphorylation is suppressed by AAP. If the studies fail to reveal that STAT3 is an important molecule mediating the anti-CSC activity of AAP, AAP effects on WNK1 will be evaluated. Immune-blotting will be used. Knock out and rescue experiments will be used to examine whether WNK1 is a putative target of AAP.

AAP does not inhibit STAT3 via direct binding at K626. Based on results described above, it is likely that AAP inhibits STAT3 via direct binding to the SH2 domain. However, if AAP does not directly bind to STAT3, then indirect inhibition of STAT3 will be evaluated. JNK, ERK, and protein kinase C have been shown to regulate S727 activity/phosphorylation on STAT3 (Johnson D E et al. Nat Rev Clin Oncol. 2018; 15 (4): 234-48). As described herein, AAP has no effect on ERK phosphorylation. To evaluate the possibility of indirect inhibition of S727 on STAT3, phosphorylation of JNK and protein kinase C will be tested in response to AAP in both H460 and A549 cells using Western blots. It will be further determined if AAP alters mTOR levels in NSCLC cell lines, given that mTOR has previously been shown to be a positive regulator of STAT3 S727 activity (Zhou J, et al. Proc Natl Acad Sci USA. 2007; 104 (41): 16158-63). To evaluate the possibility of alternative mechanisms of inhibition of Y705 phosphorylation by AAP, the effects of AAP on proteins upstream from STAT3 in the IL6/JAK/STAT3 signaling axis will be assessed. It will be determined if AAP inhibits phosphorylation of JAK2 via immunoblotting. It will also be further evaluated if AAP acts on the gp130 complex by using shRNA to genetically knockdown gp130 and determine if AAP continues to inhibit STAT3 Y705 in these mutant cells. If AAP no longer inhibits STAT3 phosphorylation at Y705 in gp130 silenced cells, then AAP inhibits STAT3 via acting on the upstream signaling molecule gp130.

### Example 12: Determine if High Dose AAP has Synergistic Anti-CSC Activity with Chemotherapy and Targeted Therapy

Based on the clinical case report of the successful treatment of a cisplatin-refractory patient with cisplatin, AAP and delayed NAC rescue (Kobrinsky N L, et al. Pediatr Blood Cancer. 2005; 45 (2): 222-5), the efficacy of cisplatin in combination with AAP was evaluated. The results demonstrated increased anti-tumor activity of AAP combined with cisplatin relative to either treatment alone in a variety of tumor models (Wu Y J, et al. Anticancer Res. 2013; 33 (6): 2391-400; Neuwelt A J, et al. Neoplasia. 2009; 11 (10): 1003-11; and Neuwelt A J, et al. Pediatr Blood Cancer. 2014; 61 (1): 120-7). It has shown that ovarian cancer xenografts treated with cisplatin monotherapy demonstrate initial tumor shrinkage, followed by regrowth. However, when the ovarian cancer bearing rat xenografts are treated with a single dose of cisplatin in combination with AAP, the tumor shrinkage is durable, and the tumor does not regrow in size (Wu Y J, et al. Anticancer Res. 2013; 33 (6): 2391-400). It has also shown that high dose AAP combined with paclitaxel leads to enhanced anti-tumor efficacy in pre-clinical models (Wu Y J, et al. Anticancer Res. 2013; 33 (6): 2391-400). Thus, it will be tested whether AAP synergizes with standard chemotherapeutic drugs used in NSCLC, e.g., carboplatin and gemcitabine.

STAT3 mediates cisplatin resistance in multiple cancer histologies (Zhu X, et al. Oncotarget. 2017; 8 (24): 39154-66; and Gu F, et al. Oncol Rep. 2010; 23 (3): 671-6). Cisplatin leads to a time and dose-dependent activation of the SRC-JAK2-STAT3 axis resulting in increased expression of STAT3 regulated anti-apoptotic molecules, particularly Bcl-xL. Treatment of NSCLC cells concurrently with cisplatin and the Bcl-XL inhibitor ABT-737 results in synergistic reduction in tumor cell viability as objectively determined using the Chou-Talalay combination index method (Kim E Y, et al. Neoplasia. 2017; 19 (4): 354-63).

In addition to mediating resistance to chemotherapy, emerging data suggests that STAT3 is involved in acquired resistance to tyrosine kinase inhibitor (TKI) therapy in oncogene-driven NSCLC (Lee H J, et al. Cancer Cell. 2014; 26 (2): 207-21). Treatment of EGFR mutant lung cancer with the EGFR inhibitor afatinib leads to increased levels phosphorylation of STAT3 leading to treatment resistance that is reversed with concurrent treatment with a STAT3 phosphorylation inhibitor (Codony-Servat C, et al. Oncotarget. 2017; 8 (29): 47305-16).

Thus, it will be tested whether AAP will synergize with chemotherapy (in NSCLC without a targetable driver mutation) and TKI therapy (in driver mutation-positive NSCLC) via a STAT3 dependent mechanism.

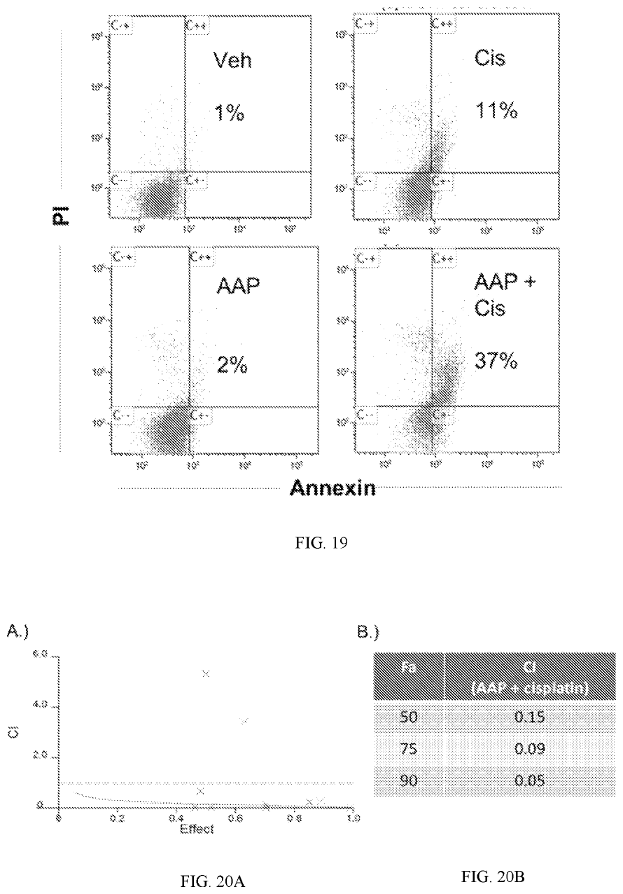

To evaluate for synergy between cisplatin and AAP in NSCLC, H460 cells in CSC media were treated with vehicle, AAP (1 mM), and/or cisplatin (0.3 μg/mL) for 48 hours. The cells were then analyzed for annexin/PI staining using flow cytometry. A substantial increase in late apoptotic (PI+Annexin+) cells was observed with combination AAP/cisplatin treatment relative to either drug alone (FIG. 19).

To determine if AAP and cisplatin lead to synergistic inhibition of spheroid formation, a primary spheroid growth assay was performed (Patel N J, et al. Oncotarget. 2016; 7 (51): 84608-22). Five hundred tumor cells were plated in CSC media in each well of a 96-well plate and treated with increasing doses of AAP and/or cisplatin. Four days later, the number of spheroids per well were counted.

Results of spheroid growth inhibition (AAP and cisplatin alone and together) was analyzed using Calcusyn® 2.0 software. CI values were calculated at Fa50/75/90 (FIG. 20). Weighted CI (CIwt=(CI50+2CI75+3C190)/6) (Chou T C. Pharmacol Rev. 2006; 58 (3): 621-81; and Chou T C. Cancer Res. 2010; 70 (2): 440-6) was used to give greater importance to synergism at higher fraction affected, which is important for anti-cancer therapy evaluation (Chou T C. Pharmacol Rev. 2006; 58 (3): 621-81). Results demonstrated a weighted CI value of 0.06, suggesting a high degree of synergy between AAP and cisplatin in H460 cells.

Methods. In vitro analysis of anti-CSC efficacy of AAP combined with standard therapy for NSCLC. The determination of whether chemotherapy/TKI therapy and AAP are synergistic in their anti-CSC activity will be made using Calcusyn analysis Chou T C, et al. J Natl Cancer Inst. 1994; 86 (20): 1517-24). The chemotherapeutic drugs that will be evaluated include cisplatin, carboplatin, gemcitabine and paclitaxel. These drugs were chosen because they are widely used in the management of NSCLC patients and have resistance mechanisms that appear to be dependent on STAT3 signaling (Ioannou N, et al. Int J Oncol. 2016; 48 (3): 908-18; and Huang W L, et al. Mol Cancer. 2010; 9:309). Synergy between AAP and TKI therapy in NSCLC with a targetable driver mutation will also be evaluated. The STAT3 constructs to be evaluated (for each cell line) are Parental, STAT3C, STAT3 WT, and STAT3 K626A. The relevant STAT3 constructs for the EGFR and EML4-ALK cell lines will be generated using similar methodology as described herein. The cell lines and treatments that will be evaluated are: EGFR: H1975 (L858R and T790M) and CUTO25 (p.E746_A750del) using AAP and osimertinib; EML4-ALK: CUTO29 (EML4 (exon 6) ALK (exon 19)) and CUTO 34 (EML4 (exon 6) ALK (exon 20) using AAP and brigatinib; and KRAS (A549, H460) using APP and carboplatin, cisplatin, paclitaxel, and gemcitabine)

Dose response curves for spheroid growth inhibition for each drug alone or in combination will be determined. Eight to ten data points for the combination studies spanning 5-log concentration range for each of the drugs will be carried out. Data will be analyzed using Calcusyn® 2.0 software. CI values will be calculated at Fa50/75/90 and weighted CI (CIwt=(CI50+2CI75+3CI90)/6) (Chou T C. Pharmacol Rev. 2006; 58 (3): 621-81; and Chou T C. Cancer Res. 2010; 70 (2): 440-6), for each of the combinations in individual cell lines will be determined. Cutoff of CI<0.5 (strong synergism) in >50% of the cells tested (at least 6/10) will be used to assign the combination the tag of ‘optimal anti-CSC combination’. CI<0.8 will be considered synergistic. Additional data on dose reduction index and median effect curves vs. CI will be obtained (Chou T C. Pharmacol Rev. 2006; 58 (3): 621-81; and Chou T C. Cancer Res. 2010; 70 (2): 440-6)).

It is expected that AAP will synergize with TKI therapy and chemotherapy in a STAT3 dependent mechanism. If synergism is reversed in STAT3 K626A, then which prevents AAP effects on STAT3 are prevented via reversal of binding affinity. STAT3C cells are expected to have reduced sensitivity to chemotherapy/TKI therapy and not demonstrate synergy with AAP because STAT3C dimerization and activation occurs independent of STAT3 phosphorylation status.

The mechanism of synergy between AAP and chemotherapy/targeted therapy will be evaluated+/−AAP on STAT3 signaling. The cell lines to be evaluated are disclosed herein, along with the treatment that is to be administered (alone or in combination with AAP). The experiments will be performed on spheroids grown in CSC medium. AAP concentration of 1 mM will be used. Dosing of chemotherapy/TKI will be roughly the IC50 as determined by the spheroid growth experiments disclosed herein.

The effects of chemotherapy/TKI therapy on STAT3 phosphorylation will be determined. NSCLC cells will be treated with chemotherapy/targeted therapy+/−AAP for 24 hours. The cells will then be collected and analyzed for pSTAT3 levels by Western blotting.

The effects of chemotherapy/TKI therapy on STAT3 nuclear translocation will also be determined. NSCLC cells will be treated with chemotherapy/targeted therapy+/−AAP for 24 hours. Immunofluorescence will be performed by fixing cells in 4% paraformaldehyde for 15 min, permeabilized with 0.3% Triton X-100 and blocked in PBS containing 2% bovine serum albumin (BSA) for 30 min. The samples will then be incubated with primary antibody to pSTAT3 or total STAT3 overnight. The cells will then counterstained with the respective secondary antibody conjugated with Alexa Fluor 484 or 594 (Molecular Probes, Eugene, OR) and Hoechst nuclei stain.

The effects of chemotherapy/TKI therapy on transcription of STAT3-dependent CSC-related genes (CD133) and anti-apoptotic genes (Bcl-xL) will be determined. NSCLC cells will be treated with chemotherapy/targeted therapy+/−AAP for 24 hours. The cells will then be collected and subjected to RNA analysis (qPCR) and protein analysis (Western blotting) of CD133 and Bcl-xL.

If synergy of AAP with chemotherapy/targeted therapy is mediated by Bcl-xL, Bcl-XL may mediate resistance to treatment of NSCLC (Shen Q, et al. Cell Death Dis. 2018; 9 (10): 986). If these experiments yield results showing that Bcl-xL may mediate synergy between AAP and chemotherapy/targeted therapy, then rescue experiments will be performed using an expression vector (Addgene #46972) in parental cells to evaluate for reversal of synergism.

The effects of whether chemotherapy/targeted therapy synergizes with high dose AAP against xenografts in vivo was determined. Cell line derived xenograft model was used. To determine if STAT3 mediates potential synergy between AAP and TKI/chemotherapy. cell-line derived xenograft experiments will be performed. In vivo growth experiments will analyze mice xenografts (using NCr mice) containing H460 Parental, STAT3 WT and STAT3 K626A Tumor cells will be flow-sorted and 10\hat{ }6 CD133+ tumor cells will be injected into mice on day 0 subcutaneously. Mice will be treated with vehicle, cisplatin (2.5 mg/kg IV (Oliva P, et al. Br J Cancer. 2012; 107 (2): 360-9)), AAP (350 mg/kg IP)+NAC (100 mg/kg IP), NAC alone, AAP+NAC+cisplatin, paclitaxel (10 mg/kg iv) Oliva P, et al. Br J Cancer. 2012; 107 (2): 360-9), or paclitaxel+AAP+NAC. Treatment will initiate on day 8 and be once per week for 4 weeks. Cisplatin and paclitaxel concentrations are chosen based on published tolerable doses that achieve partial tumor growth inhibition to allow for synergism analysis Oliva P, et al. Br J Cancer. 2012; 107 (2): 360-9). AAP and NAC doses are chosen based on experiments and data described herein.

The experiment will be repeated using H1975 EGFR mutant cells. The treatment groups will be vehicle, osimertinib (5 mg/kg daily PO (Ballard P, et al. Clin Cancer Res. 2016; 22 (20): 5130-40)), AAP (350 mg/kg weekly IP)+NAC (100 mg/kg weekly IP), NAC, or osimertinib+AAP+NAC. Osimertinib dose was chosen based on established tolerability and partial efficacy in mouse models to enable synergism analysis (Ballard P, et al. Clin Cancer Res. 2016; 22 (20): 5130-40).

Eight mice per group (alpha=0.05, and power-0.8) will be used. Mice will be 8-weeks old at start of experiment and 50% male and 50% female. NCr nude mice are a standard athymic model used for growth of human tumors and will be used. Tumor size will be assessed twice per week using digital calipers, and final size noted 35 days after tumor implantation. Tumors will be weighed at the time of animal sacrifice.

PDX experiment. A human patient derived xenograft (PDX) experiment will be performed in order to assess the efficacy of AAP combined with standard of care using tumor models that have been unaffected by years of passage in plastic and serum. PDX models of NSCLC will be obtained and grown in NSG mice (8 weeks old, 50% male and 50% female). NSG mice lack mature T, B, and functional NK cells, and are a good model for PDX experiments. PDX models can be used for the study of CSCs because PDX tumors have a heterogenous tumor microenvironment that includes a sub-population of tumor-initiating CSCs (Jahchan N S, et al. Cell Rep. 2016; 16 (3): 644-56). The PDX models to be evaluated will include a KRAS mutant cell line as well as a tumor with an EGFR activating mutation. Cryovials containing human NSCLC PDX tissue will be thawed at 37 degrees Celsius. The PDX tissue will be incubated in DMSO-free RPMI 1640 medium. The PDX will then be placed on the end of an 11-gauge trocar needle, and 100 μL of Matrigel will added to the PDX tissue. The PDX tumor will then be implanted subcutaneously between the shoulder blades of NSG mice that are 6 weeks old. The PDX tumors will be used at low passage (<5) and will be examined using morphology and directed sequencing (of driver mutation) to assure no generational drift from the parental tumor. Both male and female mice will be used in order to assure that results apply to both sexes. Tumors will be allowed to grow to 100 mm³ prior to treatment initiation. Treatment groups will be vehicle, NAC (100 mg/kg IP), AAP (350 mg/kg IP)+NAC, Cisplatin (2.5 mg/kg IV), Cisplatin+NAC, and Cisplatin+AAP+NAC, Paclitaxel (10 mg/kg), and Paclitaxel+AAP+NAC. In EGFR mutant model, the treatments will be Vehicle, osimertinib (25 mg/kg daily PO (Ballard P, et al. Clin Cancer Res. 2016; 22 (20): 5130-40)), AAP (350 mg/kg weekly IP)+NAC (100 mg/kg weekly IP), NAC, or osimertinib+AAP+NAC. Treatment will be weekly for 4 weeks. Eight mice will be used for tumor growth studies (alpha=0.05, and power-0.8). Tumor size will be assessed twice per week using digital calipers, and final size noted 35 days after tumor implantation. Tumors will be weighed at the time of animal sacrifice.

Relative timing of cisplatin and NAC. Sodium thiosulfate (STS), which like NAC is an anti-oxidant thiol, is the clinical antidote for cisplatin toxicity. When administered concurrently with cisplatin, STS reverses the anti-tumor efficacy of cisplatin. It has been shown that STS delivered at 6-hour delayed time-points does not reverse the anti-tumor efficacy of cisplatin (Harned T M, et al. Clin Cancer Res. 2008; 14 (2): 533-40)), results that were subsequently validated in a phase III international randomized trial (Harned T M, et al. Clin Cancer Res. 2008; 14 (2): 533-40). Similarly, NAC can be administered at 4-hour delayed time-points without compromising cisplatin anti-tumor efficacy (Muldoon L L, et al. J Neurooncol. 2015; 121 (3): 433-40). Thus, in the above studies AAP will be administered concurrently with NAC and both drugs will be administered 4-hours after cisplatin.

Correlative analysis. Following 4 weeks of treatment, the mice (both the PDX and cell-line derived models) will be sacrificed 3 days after final drug treatment for correlative analysis. Testing for in vivo intra-tumoral pSTAT3 levels will be performed via Western blotting and IHC. Western blotting and flow cytometry will be performed to assess the expression of proteins that are downstream of nuclear STAT3, including cyclin D1, MCL-1, and survivin. The relative intensity of individual protein bands of p-STAT3 (Y705 and S727) on immunoblots will be quantified using UN-SCAN-IT gel software 6.1 (Silk Scientific., Orem UT). Data will be presented as mean±SD (n=3).

For ex vivo analysis, tumor samples will be resected from animals upon sacrifice (3 days after final drug treatment). Tumors will be digested in collagenase at 37° C. for 30 min and filtered through 60 μm strainer to obtain single cell suspension. Tumor cells will be flow sorted to isolate CSCs for analysis. CSCs will be analyzed both separately and together (e.g., analyzing both the bulk tumor and CD133+ cells isolated with flow-sorting). Self-renewal will be evaluated using spheroid formation of a single cell suspension of isolated tumor cells (testing for 1°→4° spheroids). Apoptosis will be evaluated using annexin staining for analysis by flow cytometry. CSC-related molecular changes will also be evaluated. Expression of CSC markers (ALDH1, CD133, CD44) will be tested using flow cytometry and qPCR. CSC-associated genes (OCT4, SOX2, NANOG) will be evaluated using qPCR.

Evaluation of tumorigenic capacity; in vivo limiting dilution assay. Cells isolated from tumors following the PDX tumor growth studies will also be analyzed via an in vivo limiting dilution assay. Isolated tumor cells will be flow sorted to isolate CD133+CSCs. CSCs will be injected into tumor-naïve NSG mice, injecting 2,000, 10,000, 50,000, and 100,000 cells/mouse (Boothello R S, et al. Mol Cancer Ther. 2019; 18 (1): 51-61). Tumor size will be assessed twice per week using digital calipers, and final size noted 35 days after tumor implantation. Tumors will be weighed at the time of animal sacrifice. Four mice per condition will be used. Tumor initiating cell frequency and confidence intervals will be calculated using ELDA software (Boothello R S, et al. Mol Cancer Ther. 2019; 18 (1): 51-61).

Data analysis and results. Synergism in the in vivo experiments will be calculated using assessment of fractional tumor volumes (FTV) (Yu D C, et al. Cancer Res. 2001; 61 (2): 517-25)). The FTV will be calculated by dividing the experimental tumor volume by the vehicle tumor volume. The expected FTV of the combined treatment is the FTV of treatment #1 multiplied by the FTV of treatment #2. A ratio >1 of the expected FTV divided by the observed FTV of the combined treatment suggests synergy.

It is expected that high dose AAP will synergize with chemotherapy (in KRAS mutant cell lines) and targeted therapy (in EGFR mutant models) via a mechanism involving STAT3 inhibition in tumor cells. A mechanistic role for STAT3 in the efficacy of combined treatment with AAP and chemotherapy/targeted therapy would be supported if AAP combined with chemotherapy has decreased synergism in STAT3 K626A cells relative to STAT3 WT cells in vitro and in vivo.

It is expected that AAP/NAC will demonstrate anti-CSC activity in vivo. When tumor cells isolated from PDX-bearing mice that had been treated with AAP/NAC are re-implanted into separate mice, the CSCs will have impaired tumorigenic capacity as determined by an in vivo limiting dilution experiment.

NAC antagonizes AAP effectiveness. In the initial phase 1 studies of high dose AAP, NAC rescue was administered at delayed time-points (Kobrinsky N L, et al. Cancer Invest. 1996; 14 (3): 202-10; and Kobrinsky N L, et al. Pediatr Blood Cancer. 2005; 45 (2): 222-5). This is because NAC was thought to be an “antagonist” to the anti-tumor effects of high dose AAP (Wu G Y, et al. Hepatology. 1985; 5 (5): 709-13). The data disclosed herein shows that NAC does not compromise the anti-STAT3 or anti-CSC activity of high dose AAP. In some aspects, delayed administration of NAC will still provide hepato-protection from AAP-induced toxicity (albeit to a lesser degree then concurrent NAC (James L P, et al. Toxicol Sci. 2003; 75 (2): 458-67)). In some aspects, the relative timing of administration of cisplatin, AAP and NAC in the animal studies can be adjusted. For instance, a similar sequence as was used in the clinical case report of this regimen (James L P, et al. Toxicol Sci. 2003; 75 (2): 458-67) can be used; concurrent cisplatin and AAP followed 6-8 hours later by delayed NAC rescue.

NAC antagonizes cisplatin effectiveness. As described herein, NAC administered 4 hours after cisplatin does not compromise cisplatin efficacy in pre-clinical models (Muldoon L L, et al. J Neurooncol. 2015; 121 (3): 433-40). In some aspects, free radical independent AAP rescue agents including fomepizole (Akakpo J Y, et al. 4-Hum Exp Toxicol. 2018; 37 (12): 1310-22) or heparan sulfate octadecasaccharide (Arnold K, Xu Y, Sparkenbaugh E M, Li M, Han X, Zhang X, et al. Design of anti-inflammatory heparan sulfate to protect against acetaminophen-induced acute liver failure. Sci Transl Med. 2020; 12 (535)). can be used.

STAT3 is not the mediator of synergy between AAP and standard of care NSCLC treatments. In some aspects, RNA sequencing can be used to identify alternative targets of AAP in vivo. To determine the effects of AAP on tumor cells in vivo, without analyzing the confounding presence of mouse non-malignant immune and endothelial cells in the tumor stroma, GFP-positive mice can be implanted with GFP-negative tumor cells. After tumors develop (500 mm³), the mice will be treated with vehicle or AAP (350 mg/kg). 24-hours after treatment the mice will be sacrificed and the tumors harvested. Tumor cells will be isolated using flow-sorting of GFP-negative cells. The purified tumor cells will then be analyzed via RNA-sequencing to evaluate for alternative targets of AAP that may mediate synergy with chemotherapy and targeted therapy. Genes whose expression is altered by AAP treatment in vivo and have known effects in mediating sensitivity or resistance to standard NSCLC treatments will be further studied with knockdown/overexpression experiments similar to as described above in the methods section.

Study of AAP on the tumor immune micro-environment. The targeting of STAT3 is an immunotherapeutic approach. The IL6/STAT3 pathway is known to suppress antigen presentation of dendritic cells, leading to impaired anti-tumor immunity (Melillo J A, et al. J Immunol. 2010; 184 (5): 2638-45). Selective pulmonary knockdown of STAT3 in mouse models of urethane-induced carcinogenesis results in smaller tumors with a more inflamed phenotype relative to tumors grown in wild type mice. STAT3 knockdown tumors are characterized by a gene expression profile rich in inflammatory cytokines, including interferon gamma (Melillo J A, et al. J Immunol. 2010; 184 (5): 2638-45). Importantly, an interferon gamma gene signature has emerged as a means of predicting response to PD-1 immunotherapy in patients with multiple tumor types (Melillo J A, et al. J Immunol. 2010; 184 (5): 2638-45). In some aspects, the effects of AAP on the tumor immune microenvironment will be investigated using syngeneic mouse models of NSCLC. Further, synergism between AAP and immune stimulating therapies (e.g., PD-1 antibodies) will be evaluated in pre-clinical models. LLC cells, a syngeneic mouse NSCLC line that forms tumors in immune competent C57bl/6 mice, can be used to study of the tumor immune microenvironment (Bullock B L, et al. Life Sci Alliance. 2019; 2 (3)). AAP inhibits STAT3 phosphorylation in LLC cells.

Clinical translation. FDA approved (e.g., cisplatin, AAP, and NAC) will be studies as described herein. A clinical trial can be designed that will evaluate the efficacy of chemotherapy combined with AAP and NAC for the treatment of patients with advanced lung cancer. Importantly, translational research using thiols (STS and NAC) to protect against cisplatin-induced toxicities such as ototoxicity and nephrotoxicity has been carried out (Harned T M, et al. Clin Cancer Res. 2008; 14 (2): 533-40; Brock P R, et al. N Engl J Med. 2018; 378 (25): 2376-85; and Muldoon L L, et al. J Neurooncol. 2015; 121 (3): 433-40). In some aspects, a regimen of AAP, cisplatin and NAC may have increased efficacy (via AAP chemo-enhancement) and reduced toxicity (via NAC chemo-protection) relative to cisplatin monotherapy.

Statistical methods. For in vitro studies, the Student t-test will be used for the comparison of measurable variants between two groups. One- or two-way analysis of variance (ANOVA) followed by the Tukey Honest Significance Difference test will be used to evaluate differences among more than three groups and experimental conditions, as appropriate. P<0.05 will be considered statistically significant. Significance calculations will be performed using GraphPad Prism 9 Software. For tumor growth studies, with a two-sided alpha=0.05, and power-0.8, we will use 7 animals per group in order to detect a tumor size difference of 33% or greater. Assuming a rate of attrition 10%, we will need N=8 animal per treatment group for tumor growth studies.

In vitro studies will be repeated for three independent experiments in order to establish reproducibility. Mechanistic studies will be performed in at least two distinct NSCLC cell lines, A549 and H460, to enhance the generalizability of the findings. Independent and complementary methods will be used, for instance, in the binding studies, both spectrofluorimetry and microscale thermopheresis experiments will be used to verify the validity of the findings using orthogonal methods. To validate the anti-CSC effects of high dose AAP, multiple independent experiments will be performed to evaluate for CSC phenotype, including tumorgenicity (limiting dilution assays), self-regeneration (primary->tertiary spheroids), and surface marker expression (CD133, CD44, etc).

What is claimed is:

1. A composition comprising: acetaminophen or an analog thereof; and a CYP2E1 inhibitor.

2. The composition of claim 1, further comprising a pharmaceutical acceptable carrier.

3. The composition of claim 1, wherein the CYP2E1 inhibitor is fomepizole, propylene glycol, disulfiram, indazole, diallyl sulfide, clotrimazole, or isoniazide.

4. The composition of claim 1, wherein the acetaminophen or the analog thereof is Kp-1199, N-[2-(3,4-dihydroxyphenil)ethyl]-2-[[2-(4-hydroxyanilino)-2-oxo-ethyl]sulfamoyl]bensamide, 2-[[2-(40hydroxyanilino)-2-oxo-ethyl]sulfamoyl]-N-methylbenzamide, 5-amino-2-methoxyphenol, or 5-aminoindazole.

5. The composition of claim 1, wherein the acetaminophen or the analog thereof is present in amount of at least 0.1-400 g.

6. The composition of claim 1, wherein the composition is an aqueous form.

7. The composition of claim 1, further comprising an antioxidant.

8. The composition of claim 7, wherein the antioxidant is N-acetylcysteine, sodium thiosulfate, or amifostine.

9. A pharmaceutical liquid composition comprising: acetaminophen or an analog thereof; and a CYP2E1 inhibitor.

10. The pharmaceutical liquid composition of claim 9, further comprising a pharmaceutical acceptable carrier.

11. The pharmaceutical liquid composition of claim 10, wherein the carrier is saline.

12. The pharmaceutical liquid composition of claim 9, wherein the CYP2E1 inhibitor is fomepizole, propylene glycol, disulfiram, indazole, diallyl sulfide, clotrimazole, or isoniazide.

13. The pharmaceutical liquid composition of claim 9, wherein the acetaminophen or the analog thereof is Kp-1199, N-[2-(3,4-dihydroxyphenil)ethyl]-2-[[2-(4-hydroxyanilino)-2-oxo-ethyl]sulfamoyl]bensamide, 2-[[2-(40hydroxyanilino)-2-oxo-ethyl]sulfamoyl]-N-methylbenzamide, 5-amino-2-methoxyphenol, or 5-aminoindazole.

14. The pharmaceutical liquid composition of claim 9, wherein the acetaminophen or the analog thereof is present in amount of at least 0.1-400 g.

15. The pharmaceutical liquid composition of claim 9, further comprising an antioxidant.

16. The pharmaceutical liquid composition of claim 15, wherein the antioxidant is N-acetylcysteine, sodium thiosulfate, or amifostine.

17. The pharmaceutical liquid composition of claim 9, wherein the pharmaceutical liquid composition is aqueous.

18. The pharmaceutical liquid composition of claim 9, wherein 0.1 mL to 10 L of the composition provides a therapeutically effective dose.

19. The pharmaceutical liquid composition of claim 9, wherein the pH of the pharmaceutical liquid composition ranges from 3 to 11.

20. The pharmaceutical liquid composition of claim 9, further comprising an excipient and purified water.

21. The pharmaceutical liquid composition of claim 20, wherein the excipient is one or more of a pH adjuster, a stabilizer, a preservative, a sweetener, and a fragrance ingredient.

22. The pharmaceutical liquid composition of claim 21, wherein the pH adjuster is an alkalizing agent.

23. The pharmaceutical liquid composition of claim 22, wherein the alkalizing agent is one or more of sodium hydroxide, potassium hydroxide, sodium bicarbonate, ammonia solution, potassium citrate, triethanolamine, and sodium citrate.

24. A method of reducing or inhibiting growth of a tumor in a subject, the method comprising administering to the subject a therapeutically effective amount of the composition of any of claims 1 to 8 or the pharmaceutical composition of any of claims 9 to 23.

25. A method of reducing or inhibiting growth of a tumor in a subject, the method comprising administering to the subject a therapeutically effective amount of acetaminophen or an analog thereof and a therapeutically effective amount of a CYP2E1 inhibitor.

26. The method of claim 25, wherein the therapeutically effective amount of acetaminophen is administered to the subject, concurrently, simultaneously, or sequentially with the therapeutically effective amount of a CYP2E1 inhibitor.

27. The method of claim 24 or 25, wherein size of the tumor or the level of one or more tumor markers is reduced by at least 10%.

28. The method of claim 24 or 25, wherein the growth of the size of the tumor is less than 10% after the administration of the composition or the pharmaceutical composition compared to the growth of the size of the tumor before administration of the composition or the pharmaceutical composition.

29. The method of claim 24 or 25, wherein the method reduces the growth rate of the level of one or more tumor markers by at least 10% after the administration of the composition or the pharmaceutical composition compared to the growth rate of the one or more tumor markers before administration of the composition or the pharmaceutical composition.

30. A method of inducing apoptosis of a cancer cell, the method comprising administering to a subject in need thereof, a therapeutically effective amount of the composition of any of claims 1 to 8 or the pharmaceutical composition of any of claims 9 to 23.

31. A method of inducing apoptosis of a cancer cell, the method comprising administering to the subject a therapeutically effective amount of acetaminophen or an analog thereof and a therapeutically effective amount of a CYP2E1 inhibitor.

32. The method of claim 31, wherein the therapeutically effective amount of acetaminophen is administered to the subject, concurrently, simultaneously, or sequentially with the therapeutically effective amount of a CYP2E1 inhibitor.

33. A method of treating cancer in a subject, the method comprising administering to the subject a therapeutically effective amount of the composition of any of claims 1 to 8 or the pharmaceutical composition of any of claims 9 to 23.

34. A method of treating cancer in a subject, the method comprising administering to the subject a therapeutically effective amount of acetaminophen or an analog thereof and a therapeutically effective amount of a CYP2E1 inhibitor.

35. The method of claim 34, wherein the therapeutically effective amount of acetaminophen is administered to the subject, concurrently, simultaneously, or sequentially with the therapeutically effective amount of a CYP2E1 inhibitor.

36. The method of any of claims 24-35, further comprising administering one or more cancer therapeutic agents.

37. The method of any of claims 24-36, wherein the method reduces or prevents acetaminophen-induced liver toxicity.

38. The method of any of claims 24-37, wherein the method reduces or prevents an increase in alanine transaminase levels compared to the alanine transaminase levels before administration of the composition or pharmaceutical composition.

39. The method of any of claims 24-38, wherein the subject has cancer.

40. The method of claim 39, wherein the cancer is lung cancer, breast cancer, pancreatic cancer, esophageal cancer, colon cancer, prostate cancer or liver cancer.

41. The method of any of claims 24-36, wherein the composition or the pharmaceutical composition is administered parentally or orally.

42. The method of claim 41, wherein the parental administration is intravenous, subcutaneous, intramuscular or direct injection.
